SEQUENCE LISTING (42 sequences)

SEQ ID NO: 1 - protein, 151 aa, Hevea brasiliensis
MASVEVESAATALPKNETPEVTKAEETKTEEPAAPPASEQETADATPEKEEPTAAPAEPEAPAPETEKAEEVEKIEKTEEPAPEADQTTPEEKPAEPEPVAEEEPKHETKETETEAPAAPAEGEKPAEEEKPITEAAETATTEVPVEKTEE

SEQ ID NO: 2 - protein, 20 aa, Hevea brasiliensis
MASVEVESAATALPKNETPE

SEQ ID NO: 3 - protein, 20 aa, Hevea brasiliensis
ATALPKNETPEVTKAEETKT

SEQ ID NO: 4 - protein, 20 aa, Hevea brasiliensis
PEVTKAEETKTEEPAAPPAS

SEQ ID NO: 5 - protein, 20 aa, Hevea brasiliensis
KTEEPAAPPASEQETADATP

SEQ ID NO: 6 - protein, 20 aa, Hevea brasiliensis
ASEQETADATPEKEEPTAAP

SEQ ID NO: 7 - protein, 20 aa, Hevea brasiliensis
TPEKEEPTAAPAEPEAPAPE

SEQ ID NO: 8 - protein, 20 aa, Hevea brasiliensis
APAEPEAPAPETEKAEEVEK

SEQ ID NO: 9 - protein, 20 aa, Hevea brasiliensis
PETEKAEEVEKIEKTEEPAP

SEQ ID NO: 10 - protein, 20 aa, Hevea brasiliensis
EKIEKTEEPAPEADQTTPEE

SEQ ID NO: 11 - protein, 20 aa, Hevea brasiliensis
APEADQTTPEEKPAEPEPVA

SEQ ID NO: 12 - protein, 20 aa, Hevea brasiliensis
EEKPAEPEPVAEEEPKHETK

SEQ ID NO: 13 - protein, 20 aa, Hevea brasiliensis
VAEEEPKHETKETETEAPAA

SEQ ID NO: 14 - protein, 20 aa, Hevea brasiliensis
TKETETEAPAAPAEGEKPAE

SEQ ID NO: 15 - protein, 20 aa, Hevea brasiliensis
AAPAEGEKPAEEEKPITEAA

SEQ ID NO: 16 - protein, 20 aa, Hevea brasiliensis
AEEEKPITEAAETATTEVPV

SEQ ID NO: 17 - protein, 20 aa, Hevea brasiliensis
PITEAAETATTEVPVEKTEE

SEQ ID NO: 18 - protein, 19 aa, Hevea brasiliensis
TPEKEEPTAAPAEPEAPAP

SEQ ID NO: 19 - protein, 18 aa, Hevea brasiliensis
TPEKEEPTAAPAEPEAPA

SEQ ID NO: 20 - protein, 17 aa, Hevea brasiliensis
TPEKEEPTAAPAEPEAP

SEQ ID NO: 21 - protein, 16 aa, Hevea brasiliensis
TPEKEEPTAAPAEPEA

SEQ ID NO: 22 - protein, 15 aa, Hevea brasiliensis
TPEKEEPTAAPAEPE

SEQ ID NO: 23 - protein, 14 aa, Hevea brasiliensis
TPEKEEPTAAPAEP

SEQ ID NO: 24 - protein, 13 aa, Hevea brasiliensis
TPEKEEPTAAPAE

SEQ ID NO: 25 - protein, 12 aa, Hevea brasiliensis
TPEKEEPTAAPA

SEQ ID NO: 26 - protein, 11 aa, Hevea brasiliensis
TPEKEEPTAAP

SEQ ID NO: 27 - protein, 10 aa, Hevea brasiliensis
TPEKEEPTAA

SEQ ID NO: 28 - protein, 9 aa, Hevea brasiliensis
TPEKEEPTA

SEQ ID NO: 29 - protein, 8 aa, Hevea brasiliensis
TPEKEEPT

SEQ ID NO: 30 - protein, 19 aa, Hevea brasiliensis
PEKEEPTAAPAEPEAPAPE

SEQ ID NO: 31 - protein, 18 aa, Hevea brasiliensis
EKEEPTAAPAEPEAPAPE

SEQ ID NO: 32 - protein, 17 aa, Hevea brasiliensis
KEEPTAAPAEPEAPAPE

SEQ ID NO: 33 - protein, 16 aa, Hevea brasiliensis
EEPTAAPAEPEAPAPE

SEQ ID NO: 34 - protein, 15 aa, Hevea brasiliensis
EPTAAPAEPEAPAPE

SEQ ID NO: 35 - protein, 14 aa, Hevea brasiliensis
PTAAPAEPEAPAPE

SEQ ID NO: 36 - protein, 13 aa, Hevea brasiliensis
TAAPAEPEAPAPE

SEQ ID NO: 37 - protein, 12 aa, Hevea brasiliensis
AAPAEPEAPAPE

SEQ ID NO: 38 - protein, 11 aa, Hevea brasiliensis
APAEPEAPAPE

SEQ ID NO: 39 - protein, 10 aa, Hevea brasiliensis
PAEPEAPAPE

SEQ ID NO: 40 - protein, 9 aa, Hevea brasiliensis
AEPEAPAPE

SEQ ID NO: 41 - protein, 8 aa, Hevea brasiliensis
EPEAPAPE

SEQ ID NO: 42 - protein, 10 aa, Hevea brasiliensis
EPTAAPAEPE

(19) United States
(12) Patent Application Publication
(10) Pub. No.: US 2007/0059756 A1
(43) Pub. Date: Mar. 15, 2007

(54) Immunointeractive molecules and their uses in treating subjects suffering from Hev b 5 allergies

(76) Inventor: Robyn O'Hehir, Parkville (AU)

Correspondence Address: KNOBBE MARTENS OLSON & BEAR LLP, 2040 MAIN STREET, FOURTEENTH FLOOR, IRVINE, CA, 92614, US

(21) Appl. No.: 11/595,628

(22) Filed: Nov. 8, 2006

Related U.S. Application Data
(62) Division of application No. 10/089,273, filed on Aug. 6, 2002, now abandoned.

(30) Foreign Application Priority Data
Sep. 24, 1999 (AU) PQ 3057
Jul. 24, 2000 (AU) PQ 8964

(51) Int. Cl. C12Q 1/68; C12P 21/06

(52) U.S. Cl. 435/6; 435/69.1

(57) ABSTRACT

The present invention relates generally to molecules such as peptides, polypeptides and proteins which interact immunologically with T lymphocytes in subjects having latex allergy and genetic sequences encoding same. These molecules are preferentially immunointeractive with T cells in subjects having a Hev b 5 allergy. The present invention also extends to antibodies, preferably monoclonal antibodies, directed to latex allergens and in particular to Hev b 5, and to the B cell epitopes recognised therein. The molecules of the present invention are useful in the development of diagnostic, therapeutic and prophylactic agents for conditions characterised by an aberrant, inappropriate or otherwise unwanted immune response to Hev b 5 or derivative or homologue thereof.

### FIELD OF THE INVENTION

The present invention relates generally to molecules such as peptides, polypeptides and proteins which interact immunologically with T lymphocytes in subjects having latex allergy and genetic sequences encoding same. These molecules are preferentially immunointeractive with T cells in subjects having a Hev b 5 allergy. The present invention also extends to antibodies, preferably monoclonal antibodies, directed to latex allergens and in particular to Hev b 5, and to the B cell epitopes recognised therein The molecules of the present invention are useflul in the development of diagnostic, therapeutic and prophylactic agents for conditions characterised by an aberrant, inappropriate or otherwise unwanted immune response to Hev b 5 or derivative or homologue thereof.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia

IgE mediated hypersensitivity to latex has emerged as a serious occupational health problem since the introduction of universal precautions in the mid 1980s (Slater, 1994; Sussman et al., 1991). The use of latex gloves for barrier protection may lead to sensitisation, especially of health care workers (HCW), to protein allergens present in the natural rubber latex. The prevalence of latex sensitisation amongst HCW has been reported from 8.2-22% (Grzybowski et al., 1996; Kibby and AkI, 1997; Liss et al., 1997; Brown et al., 1988; Douglas et al., 1997). Allergic reactions to latex range from urticaria, rhinoconjunctivitis, asthma (Brugnami et al., 1995), and angioedema to severe generalised anaphylaxis in some cases (Ownby et al., 1991). Since the only form of treatment available at present is allergen avoidance and symptomatic relief, there is an urgent need for the development of specific immunotherapy for this condition. Thus, the immunological characterisation of latex allergens is an important breakthrough in the development of rational curative treatments for latex allergy.

Several allergens from the rubber plant Hevea brasiliensis have been identified (Chen et al., 1996; Yeang et al., 1996; Sowka et al., 1998; Scheiner et al. 1999; Kostyal et al., 1998; Akasawa et al., 1996; Slater et al, 1996; Chen et al., 1998; Posch et al., 1998). Based on IgE binding studies, certain latex allergens seem to be preferentially recognised by particular risk groups (Hev b 1 and 3 by children with spina bifida and Hev b 5 and 6.02 by HCW (Breitender, 1998)). Hev b 5, a highly acidic and proline rich protein with a predicted predominantly random secondary structure, has been shown to react with IgE from 92% of HCW and 56% of spina bifida patients with latex allergy (Slater et al., 1996). Thus Hev b 5 has been identified as a major allergen in natural rubber latex. Interestingly, the amino acid sequence of Hev b 5 shows 46% identity to another acidic protein identified in kiwi fruit (Actinidia deliciosa) (Slater et al., 1996) and this may provide a molecular explanation for the high frequency of fruit hypersensitivity seen in latex allergic patients (Brehler et al, 1997).

The production of allergen specific IgE by B cells and release of inflammatory mediators by mast cells and eosinophils result in the effector response of allergic disease. However, it is well established that these events are orchestrated by allergen-specific CD4⁺ T cells with a Th₂-type cytokine profile. T cell reactive determinants have been reported for another major latex allergen, Hev b 1 (Raulf-Heimsoth, 1998), but not for Rev b 5. Thus, characterization of the immune response to Hev b 5 is critical in the development of specific diagnostic and immunotherapeutic methodology.

In work leading up to the present invention, the inventors have identified the human T cell epitopes of the latex allergen, Hev b 5. Although murine T cell epitopes of Hev b 5 have previously been identified in BALB/c mice, there is not necessarily a correlation between species in terms of the sharing of T or B cell epitopic regions on homologous molecules. In this regard, the inventors have confirmed that the immunodominant human Hev b 5 T cell epitopes which they have identified, and which are disclosed herein, do not correspond to the dominant murine T cell Hev b 5 epitopes. This variation between murine and human immunodominance demonstrates the difficulties associated with elucidating aspects of immune response mechanisms, such as the identification of epitopes, and highlights the novel and surprising results obtained by the inventors. The inventors have also developed monoclonal antibodies directed to Hev b 5 thereby facilitating the identification of Hev b 5 B cell epitopes. The identification of Hev b 5 T and B cell epitopes and the production of monoclonal antibodies to Hev b 5 now facilitates the development of molecules and methodology for the diagnosis and treatment of conditions characterised by the aberrant, inappropriate or otherwise unwanted immune response to Hev b 5 or derivative or homologue thereof such as latex allergy or fruit hypersensitivity.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The subject specification contains amino acid sequence information prepared using the programme PatentIn Version 2.0, presented herein after the bibliography. Each amino acid sequence is identified in the sequence listing by the numeric indicator <210> followed by the sequence identifier (eg. <210>1, <210>2, etc). The length, type of sequence (protein (PRT), etc) and source organism for each amino acid sequence is indicated by information provided in the numeric indicator fields <211>, <212> and <213>, respectively. Amino acid sequences referred to in the specification are defined by the information provided in numeric indicator field <400> followed by the sequence identifier (eg. <400>1, <400>2, etc).

One aspect of the present invention provides an isolated peptide of the formula:
X₁X₂X₃
wherein:
X₁ and X₃ may be the same or different and each is an amino acid sequence comprising from 0 to 40 naturally or non-naturally occurring amino acid residues;
X₂ is any amino acid sequence derived from or homologous to Hev b 5;
   and wherein said peptide molecule is capable of interacting with T cells and modifying T cell function when incubated with cells from subjects having a condition characterised by an aberrant, unwanted or otherwise inappropriate immune response to Hev b 5 or a derivative, homologue, analogue, mutant, chemical equivalent or mimetic of said peptide.

The present invention therefore more particularly provides an isolated peptide of the formula:
X₁X₂X₃
wherein:
X₁X₃ may be the same or different and each is an amino acid sequence comprising from 0 to 40 naturally or non-naturally occurring amino acid residues;
X₂ is an amino acid sequence of from 5 to 100 residues derived from, homologous to or contiguous with amino acids 1-151 inclusive or derivatives thereof of Hev b 5;
   and wherein said peptide molecule is capable of interacting with T cells and modifying T cell function when incubated with cells from subjects having a condition characterised by an aberrant, unwanted or otherwise inappropriate immune response to Hev b 5 or a derivative, homologue, mutant, chemical equivalent or mimetic of said peptide.

Still more particularly the present invention provides an isolated peptide of the formula:
X₁X₂X₃
wherein
X₁ and X₃ may be the same or different and each is an amino acid sequence comprising from 0 to 40 naturally or non-naturally occurring amino acid residues;
X₂ is an amino acid sequence of from 5 to 100 residues derived from, homologous to or contiguous with amino acids 37-74 or 109-146 inclusive or derivatives thereof of Hev b 5;
   and wherein said peptide molecule is capable of interacting with T cells and modifying T cell function when incubated with cells from subjects having a condition characterised by an aberrant, unwanted or otherwise inappropriate immune response to Hev b 5 or a derivative, homologue, analogue, mutant, chemical equivalent or mimetic of said peptide.

Still more particularly, X₂ is any amino acid sequence of from 5 to 100 residues derived from, homologous to or contiguous with amino acids 1-20, 19-110 and/or 109-146 inclusive or derivatives thereof of Hev b 5.

Yet more particularly, X₂ is any amino acid sequence of from 5 to 100 residues derived from homologous to or contiguous with amino acids 37-56, 46-65, 55-74, 109-128 and/or 127-146 inclusive or derivatives thereof of Hev b 5.

Most particularly, X₂ is any amino acid sequence of from 5 to 100 residues derived from, homologous to or contiguous with amino acids 46-65 and/or 109-128 inclusive or derivatives thereof of Hev b 5.

Still more particularly, X₂ is any amino acid sequence of from 5 to 100 residues derived from, homologous to or contiguous with amino acids 51-60 inclusive or derivatives thereof of Hev b 5.

In a particularly preferred embodiment, X₂ comprises a sequence of at least 5 amino acids derived from one or more of the following amino acid sequences:

ASEQETADATPEKEEPTAAP <400> 6
TPEKEEPTAAPAEPEAPAPE <400> 7
APAEPEAPAPETEKAEEVEK <400> 8
TKETETEAPAAPAEGEKPAE <400> 14
AEEEKPITEAAETATTEVPV <400> 16

More preferably, X₂ comprises a sequence of at least 5 amino acids derived from one or more of <400>7 or <400>14.

Still more preferably, X₂ comprises a sequence of at least 5 amino acids derived from <400>7.

Most preferably, X₂ comprises a sequence of at least 5 amino acids derived from the following amino acid sequence:

EPTAAPAEPE <400> 42.

Another aspect of the present invention provides an isolated peptide comprising any amino acid sequence derived from or homologues to Hev b 5 wherein said peptide molecule is capable of interacting with T cells and modifying T cell function when incubated with cells from subjects having a condition characterised by an aberrant, unwanted or otherwise inappropriate immune response to Hev b 5 or a derivative, homologue, analogue, mutant, chemical equivalent or mimetic of said peptide.

Yet another aspect of the present invention provides an isolated peptide comprising an amino acid sequence of from 5-100 residues derived from, homologues to or contiguous with amino acids 1-151 inclusive or derivatives thereof of Hev b 5 wherein said peptide molecule is capable of interacting with T cells and modifying T cell function when incubated with cells from subjects having a condition characterised by an aberrant, unwanted or otherwise inappropriate immune response to Hev b 5 or a derivative, homologue, analogue, mutant, chemical equivalent or mimetic of said peptide.

In one preferred embodiment said amino acid sequence is derived from, homologous to or contiguous with amino acids 37-74 or 109-146 inclusive or derivatives thereof of Hev b 5.

In another preferred embodiment said amino acid sequence is derived from, homologous to or contiguous with amino acids 1-20. 19-110 and/or 109-146 inclusive or derivatives thereof of Hev b 5.

In yet another preferred embodiment said amino acid sequence is derived from, homologous to or contiguous with amino acids 37-56, 46-65, 55-74, 109-128 and/or 127-146 inclusive or derivatives thereof of Hev b 5.

More preferably, said amino acid sequence is derived from, homologous to or contiguous with amino acids 46-65 and/or 109-128, and most preferably amino acids 51-60, inclusive or derivatives thereof of Hev b 5.

In another aspect said amino acid sequence comprises a sequence of at least 5 amino acids derived from one or more of the following amino acid sequences:

ASEQETADATPEKEEPTAAP <400> 6
TPEKEEPTAAPAEPEAPAPE <400> 7
APAEPEAPAPETEKAEEVEK <400> 8
TKETETEAPAAPAEGEKPAE <400> 14
AEEEKPITEAAETATTEVPV <400> 16

According to this aspect, said amino acid sequence preferably comprises a sequence of at least 5 amino acids derived from one or more of <400>7 or <400>14.

Still more preferably, said amino acid sequence comprises a sequence of at least 5 amino acids derived from <400>7.

Most preferably, said amino acid sequence comprises a sequence of at least 5 amino acids derived from the following amino acid sequence:

EPTAAPAEPE <400> 42.

Another aspect of the present invention is directed to antibodies to Hev b 5 including catalytic antibodies or derivatives, homologues, analogues, mutants, chemical equivalents or mimetics of said antibodies.

In a preferred embodiment the subject antibody is 1C10, 6F6, 3G3 or 6A10 and, more particularly, 1C10, 6F6 or 3G3 or derivative, homologue, analogue, chemical equivalent, mutant or mimetic thereof.

In another aspect there is provided an isolated peptide of the formula:
X₁X₂X₃
wherein
X₁ and X₃ may be the same or different and each is an amino acid sequence comprising from 0 to 40 naturally or non-naturally occurring amino acid residues;
X₂ is an amino acid sequence derived from, or homologous to Hev b 5
   and wherein said peptide molecule is capable of interacting with antibody from subjects having a condition characterised by an aberrant, unwanted or otherwise inappropriate immune response to Hev b 5 or a derivative, homologue, analogue, mutant, chemical equivalent or mimetic of said peptide.

Preferably, said condition is latex hypersensitivity and said antibodies are of the IgE isotype.

More particularly the present invention provides an isolated peptide of the formula:
X₁X₂X₃
wherein
X₁ and X₃ may be the same or different and each is an amino acid sequence comprising from 0 to 40 naturally or non-naturally occurring amino acid residues;
X₂ is any amino acid sequence derived from, or homologous to Hev b 5
   and wherein said peptide molecule is capable of interacting with antibodies 1C10, 6F6, 3G3 or 6A10 or a derivative, homologue, analogue, mutant, chemical equivalent or mimetic of said peptide.

In another aspect, the present invention provides a nucleic acid molecule comprising a sequence of nucleotides encoding or complementary to a sequence encoding the peptides as hereinbefore defined or a derivative, homologue or analogue thereof

In yet another aspect the present invention provides a method for the treatment and/or prophylaxis of a condition in the subject, which condition is characterised by the aberrant, unwanted or otherwise inappropriate immune response to Hev b 5, said method comprising administering to said subject an effective amount of a peptide and/or antibody as hereinbefore defined for a time and under conditions sufficient to remove or reduce the presence or function in said subject of T cells and/or antibodies directed to said Hev b 5.

Still another aspect of the present invention contemplates the use of an agent as hereinbefore defined in the manufacture of a medicament for the treatment of a condition in a mammal, which condition is characterised by an aberrant, unwanted or otherwise inappropriate immune response to Hev b 5.

In yet another further aspect, the present invention contemplates a pharmaceutical composition comprising an agent as hereinbefore defined and one or more pharmaceutically acceptable carriers and/or diluents. Said agents are referred to as the active ingredients.

Yet another aspect of the present invention relates to agents, as hereinbefore defined, when used in the method of the present invention.

Still yet another aspect of the present invention is directed to a method of diagnosing or monitoring a condition in a mammal, which condition is characterised by an aberrant, unwanted or inappropriate response to Hev b 5, said method comprising screening for Hev b 5 reactive T cells and/or antibodies utilising a peptide as hereinbefore defined.

Still another aspect of the present invention is directed to a method of qualitatively and/or quantitatively detecting Hev b 5, or peptides thereof, in a sample said method comprising screening for said Hev b 5 or peptides thereof utilising an antibody as hereinbefore defined.

In a further aspect the present invention provides diagnostic kits for use in the diagnostic methodology hereinbefore defined.

Single and three letter abbreviations used throughout the specification are defined in Table 1.

TABLE 1
Single and three letter amino acid abbreviations

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any residue | Xaa | X |

### BRIEF DESCRIPTION OF THE DRAWINGS

FIG. 1 is a schematic representation of the primary amino acid sequences of overlapping synthetic peptides, spanning the entire length of the Hev b 5 molecule. Amino acid residues are indicated with the single letter code. All peptides are 20 mers with 11 amino acid overlap except for the last two peptide that overlap by 15 residues.

FIG. 2 is an image of dot immunoblot analysis of serum IgE reactivity with latex allergens for the latex allergic patients 1-6.

FIG. 3 is a graphical representation of the proliferation of (A) PBMC and (B) latex specific T cell line of patient 2 in response to latex allergens. (A) PBMC proliferative response to LAL (0.03-100 μg/ml), rHev b 5/MBP (5-40 μg/ml), MBP (5-40 μg/ml) was assessed by ³H thymidine incorporation. Tetanus Toxoid (tet-tox) (0.3 Lf/ml) and Lymphocult-T (10 IU/ml) were included as controls. Results are expressed as cpm±SD of triplicate cultures. Cells alone in the absence of antigen were included to assess background levels of cell proliferation. (B) T cell line proliferation in the presence of Hev b 5 peptides (10,30) μg/ml), LAL (3-100 μg/ml), rHev b 5/MBP (10 μg/ml), MBP (10 μg/ml), PHA (3 μg/ml) and Lymphocult-T (10 IU/ml) was assessed by ³H-thymidine incorporation. T+APC alone. T alone and APC alone were included to assess background levels of cell proliferation.

FIG. 4 is a graphical representation of the (A) Proliferation and (B) IL-5 production by latex specific T cell line of latex allergic patient 4. (A) Proliferation of T cells in response to Hev b 5 peptides (30, 10 μg/ml), LAL (100, 30, 10, 3 μg/ml) and PHA (3 μg/ml) assessed by ³H thymidine incorporation (values shown are averages of triplicate samples with SD indicated). Background level of cell proliferation (T+APC) is indicated by the horizontal line at 1000 cpm. (B) Production of IL-5 in the culture supernatants determined by ELISA. Only the indicated peptides were tested for their ability to stimulate T cell production of IL-5. NT indicates peptides that were not tested in the IL-5 assay. Values indicate averages of duplicate samples.

FIG. 5 is a graphical representation of the comparison of human and murine T cell determinants of Hev b 5. Latex allergic patient (1-5) T cell stimulation indices ≧2.5 in response to Hev b 5 peptide stimulation are shown by the hatched bars. Previously identified murine T cell epitopes³⁰ are shown in shading.

FIG. 6 is a graphical representation of the immune response of mouse number 3 during the immunisation schedule with rHev b5/MBP.

FIG. 7 is a graphical representation of an ELISA of hybridoma supernatants.

FIG. 8 is an image of a Western blot of a rHev b 5 fusion protein digest.

FIG. 9 is an image of a Western blot of NAL.

FIG. 10 is a graphical representation of the determination of the minimal epitope of Hev b 5 p(46-65). A three week low ammoniated latex (LAL)-specific T cell line was stimulated with the Hev b 5 p(46-65) truncated peptides at two concentrations, 10 μg/ml and 30 μg/ml. Proliferation was assessed at 72 hours by ³H-thymidine incorporation in counts per minute (A). Levels of IL-5 (B) and IFN-γ (C) in culture supernatants were measured at 48 hours using a commercially available sandwich ELISA kit.

### DETAILED DESCRIPTION OF THE INVENTION

The present invention is predicated, in part, on the identification of Hev b 5 T and B cell epitopic regions and the development of monoclonal antibodies to Hev b 5. The identification of immunodominant epitopes of Hev b 5 has enabled the improvement of diagnostic methodology and the development of therapeutic and prophylactic compositions and treatment approaches for conditions such as, but not limited to, latex allergy.

In accordance with the present invention, overlapping peptides were synthesised based on the Hev b 5 amino acid sequence disclosed in <400>1. The T cell immunoreactivity of these peptides is identified in accordance with the present invention on the basis of interactivity of peripheral blood cells or T cells obtained from the peripheral blood of subjects with latex hypersensitivity. The identification of Hev b 5 B cell epitopes has been facilitated via the generation of a panel of monoclonal antibodies directed to Hev b 5. The identification and generation of those molecules thereby form the basis for a new range of diagnostic, therapeutic and prophylactic reagents and procedures.

Accordingly, one aspect of the present invention provides an isolated peptide of the formula:
X₁X₂X₃
wherein:
X₁ and X₃ may be the same or different and each is an amino acid sequence comprising from 0 to 40 naturally or non-naturally occurring amino acid residues;
X₂ is any amino acid sequence derived from or homologous to Hev b 5;
   and wherein said peptide molecule is capable of interacting with T cells and modifying T cell function when incubated with cells from subjects having a condition characterised by an aberrant, unwanted or otherwise inappropriate immune response to Hev b 5 or a derivative, homologue, analogue, mutant, chemical equivalent or mimetic of said peptide.

Hev b 5 is a protein which has been identified in the rubber plant Hevea brasiliensis. It is a highly acidic and proline rich protein with a predicted predominantly random secondary structure. It has also been shown to share homology with acidic proteins found in fruit, in particular, an acidic protein identified in kiwi fruit (Actinidia deliciosa) (Slater et al., 1996). Accordingly, reference to “Hev b 5” should be understood as including reference to all forms of Hev b 5 or derivatives, mutants, homologues, analogues, equivalents or mimetics thereof. This includes, for example, all protein forms of Hev b 5 or its functional equivalent or derivative including, for example, any isoforms which may arise from alternative splicing of Hev b 5 mRNA. It includes reference to mutants, polymorphic variants or homologues of Hev b 5, such as the homologous kiwi fruit protein detailed above. It also includes reference to analogues or equivalents of Hev b 5 such as may occur where a product which naturally comprises Hev b 5 is synthetically generated for the purpose of generating a product such as gloves. The present invention thereby provides epitopes and methods for their use in the diagnosis and treatment of any condition characterised by hypersensitivity to a Hev b 5 or Hev b 5-like molecule such as latex allergy or a fruit allergy. Preferably, said Hev b 5 comprises the sequence set forth in <400>1 or is a derivative, homologue, analogue, chemical equivalent, mutant or mimetic of said sequence.

The present invention therefore more particularly provides an isolated peptide of the formula:
X₁X₂X₃
wherein:
X₁ and X₃ may be the same or different and each is an amino acid sequence comprising from 0 to 40 naturally or non-naturally occurring amino acid residues;
X₂ is an amino acid sequence of from 5 to 100 residues derived from, homologous to or contiguous with amino acids I-151 inclusive or derivatives thereof of Hev b 5;
   and wherein said peptide molecule is capable of interacting with T cells and modifying T cell function when incubated with cells from subjects having a condition characterised by an aberrant, unwanted or otherwise inappropriate immune response to Hev b 5 or a derivative, homologue, mutant, chemical equivalent or mimetic of said peptide.

Still more particularly the present invention provides an isolated peptide of the formula:
X₁X₂X₃
wherein
X₁ and X₃ may be the same or different and each is an amino acid sequence comprising from 0 to 40 naturally or non-naturally occurring amino acid residues;
X₂ is an amino acid sequence of from 5 to 100 residues derived from, homologous to or contiguous with amino acids 37-74 or 109-146 inclusive or derivatives thereof of Hev b 5;
   and wherein said peptide molecule is capable of interacting with T cells and modifying T cell function when incubated with cells from subjects having a condition characterised by an aberrant, unwanted or otherwise inappropriate immune response to Hev b 5 or a derivative, homologue, analogue, mutant, chemical equivalent or mimetic of said peptide.

Still more particularly, X₂ is any amino acid sequence of from 5 to 100 residues derived from, homologous to or contiguous with amino acids 1-20, 19-110 and/or 109-146 inclusive or derivatives thereof of Hev b 5.

Yet more particularly, X₂ is any amino acid sequence of from 5 to 100 residues derived from homologous to or contiguous with amino acids 37-56, 46-65, 55-74, 109-128 and/or 127-146 inclusive or derivatives thereof of Hev b 5.

Most particularly, X₂ is any amino acid sequence of from 5 to 100 residues derived from, homologous to or contiguous with amino acids 46-65 and/or 109-128 inclusive or derivatives thereof of Hev b 5.

Still more particularly, X₂ is any amino acid sequence of from 5 to 100 residues derived from, homologous to or contiguous with amino acids 51-60 inclusive or derivatives thereof of Hev b 5.

Reference to “T cells” should be understood as a reference to any cell comprising a T cell receptor. In this regard, the T cell receptor may comprise any one or more of the α, β, γ or δ chains. The present invention is not intended to be limited to any particular functional sub-class of T cells although in a preferred embodiment the subject T cell is a T helper cell and still more preferably a Th2-type cell, predominantly. In this regard, reference to “modifying T cell function” should be understood as a reference to modifying any one or more functions which a T cell is capable of performing. For example, the subject function may be proliferation, differentiation or other form of cellular functional activity such as the production of cytokines. Preferably, the subject functional activity is proliferation.

In terms of modifying the function of T cells from subjects having a condition characterised by an aberrant, unwanted or inappropriate immune response to Hev b 5, it should be understood that this is not necessarily a reference to modifying the function of all the T cells in a given sample but is likely, in fact, to reflect the modification or functioning of only some of the T cells in the sample. For example, only a portion of the T helper cells in a given T cell sample may functionally respond to contact with the subject peptide. Such a partial response should be understood to fall within the scope of the present invention. It should also be understood that the T cells which are derived from the subject may be freshly harvested T cells or they may have undergone some form of in vitro or in vivo manipulation prior to testing. For example, T cell lines may have been generated from the cell sample and it is these T cell lines which then form the subject derived T cell population which is tested in accordance with the present invention. To the extent that the subject functional activity is T cell proliferation, the T cell proliferation assay is preferably performed as disclosed herein. Still more preferably, the subject modification of T cell function is the induction of a proliferation index of ≧2.5.

Reference to an “aberrant, unwanted or otherwise inappropriate” immune response should be understood as a reference to any form of physiological activity which involves the activation and/or functioning of one or more immune cells where that activity is inappropriate in that it is of an inappropriate type or proceeds to an inappropriate degree. It may be aberrant in that according to known immunological principals it either should not occur when it does so or else should occur when it does not do so. In another example, the immune response may be inappropriate in that it is a physiologically normal response but which is unnecessary and/or unwanted, such as occurs with respect to type-I hypersensitivity responses to innocuous allergens. Preferably said immune response is latex hypersensitivity.

By “latex hypersensitivity” it should be understood to mean the exhibition of clinical symptoms of IgE mediated latex hypersensitivity as tested via latex specific serum IgE which is measured using the Kallestad Allercoat EAST System (Sanofi-Pasteur Diagnostics, USA). In accordance with this test, the latex EAST score in non-allergic individuals is 0/4. Accordingly any subject exhibiting a latex EAST score greater than 0/4 should be understood as a subject exhibiting latex hypersensitivity within the context of the present invention.

In a preferred embodiment, X₂ comprises not less than about 5 and not greater than about 50 amino acid residues, more preferably not less than about 5 and not greater than about 30 amino acid residues and even more preferably not less than about 5 and not greater than about 20.

In a particularly preferred embodiment, X₂ comprises a sequence of at least 5 amino acids derived from one or more of the following amino acid sequences:

ASEQETADATPEKEEPTAAP <400> 6
TPEKEEPTAAPAEPEAPAPE <400> 7
APAEPEAPAPETEKAEEVEK <400> 8
TKETETEAPAAPAEGEKPAE <400> 14
AEEEKPITEAAETATTEVPV <400> 16

More preferably, X₂ comprises a sequence of at least 5 amino acids derived from one or more of <400>7 or <400>14.

Still more preferably, X₂ comprises a sequence of at least 5 amino acids derived from <400>7.

Most preferably, X₂ comprises a sequence of at least 5 amino acids derived from the following amino acid sequence:

EPTAAPAEPE <400> 42.

Reference to a “peptide” includes reference to a peptide, polypeptide or protein or parts thereof. The peptide may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “peptide” includes a peptide comprising a sequence of amino acids as well as a peptide associated with other molecules such a amino acids, lipids, carbohydrates or other peptides, polypeptides or proteins.

“Derivatives” include fragments, parts, portions and variants from natural, synthetic or recombinant sources including fusion proteins. Parts or fragments include, for example, active regions of the subject peptide. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence.

Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins.

Chemical and functional equivalents of the subject peptide should be understood as molecules exhibiting any one or more of the functional activities of these molecules and may be derived from any source such as being chemically synthesized or identified via screening processes such as natural product screening.

Homologues include peptides derived from species other than Hevea brasiliensis, such as peptides derived from kiwi fruit.

The derivatives include fragments having particular epitopes or parts of the entire protein fused to peptides, polypeptides or other proteinaceous or non-proteinaceous molecules.

Analogues contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 2.

TABLE 2

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Mom |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Crosslinkers can be used, for example, to stabilise 3D conformations, using homo-bifunctional crosslinkers such as the bifunctional imido esters having (CH2)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

It is possible to modify the structure of a peptide according to the invention for various purposes such as for increasing solubility, enhancing therapeutic or preventative efficacy, enhancing stability or increasing resistance to proteolytic degradation. A modified peptide may be produced in which the amino acid sequence has been altered, such as by amino acid substitution, deletion or addition, to modify immunogenicity and/or reduce allergenicity. Similarly components may be added to peptides of the invention to produce the same result.

For example, a peptide can be modified so that it exhibits the ability to induce T cell anergy. In this instance, critical binding residues for the T cell receptor can be determined using known techniques (for example substitution of each residue and determination of the presence or absence of T cell reactivity). In one example, those residues shown to be essential to interact with the T cell receptor can be modified by replacing the essential amino acid with another, preferably similar amino acid residue (a conservative substitution) whose presence is shown to alter T cell reactivity or T cell functioning. In addition, those amino acid residues which are not essential for T cell receptor interaction can be modified by being replaced by another amino acid whose incorporation may then alter T cell reactivity or T cell functioning but does not, for example, eliminate binding to relevant MHC proteins.

Such modifications will result in the production of molecules falling within the scope of “mutants” of the subject peptide as herein defined. “Mutants” should be understood as a reference to peptides which exhibit one or more structural features or functional activities which are distinct from those exhibited by the non-mutated peptide counterpart. Peptides of the invention may also be modified to incorporate one or more polymorphisms resulting from natural allelic variation and D-amino acids, non-natural amino acids or amino acid analogues may be substituted into the peptides to produce modified peptides which fall within the scope of the invention. Peptides may also be modified by conjugation with polyethylene glycol (PEG) by known techniques. Reporter groups may also be added to facilitate purification and potentially increase solubility of the peptides according to the invention. Other well-known types of modification including insertion of specific endoprotease cleavage sites, addition of functional groups or replacement of hydrophobic residues with less hydrophobic residues as well as site-directed mutagenesis of DNA encoding the peptides of the invention may also be used to introduce modifications which could be useful for a wide range of purposes. The various modifications to peptides according to the invention which have been mentioned above are mentioned by way of example only and are merely intended to be indicative of the broad range of modifications which can be effected.

Another aspect of the present invention provides an isolated peptide comprising any amino acid sequence derived from or homologues to Hcv b 5 wherein said peptide molecule is capable of interacting with T cells and modifying T cell function when incubated with cells from subjects having a condition characterised by an aberrant, unwanted or otherwise inappropriate immune response to Hev b 5 or a derivative, homologue, analogue, mutant, chemical equivalent or mimetic of said peptide.

More particularly, the present invention provides an isolated peptide comprising an amino acid sequence of from 5-100 residues derived from, homologues to or contiguous with amino acids 1-151 inclusive or derivatives thereof of Hev b 5 wherein said peptide molecule is capable of interacting with T cells and modifying T cell function when incubated with cells from subjects having a condition characterised by an aberrant, unwanted or otherwise inappropriate immune response to Hev b 5 or a derivative, homologue, analogue, mutant, chemical equivalent or mimetic of said peptide.

In one preferred embodiment said amino acid sequence is derived from, homologous to or contiguous with amino acids 37-74 or 109-146 inclusive or derivatives thereof of Hev b 5.

In another preferred embodiment said amino acid sequence is derived from, homologous to or contiguous with amino acids 1-20, 19-110 and/or 109-146 inclusive or derivatives thereof of Hev b 5.

In yet another preferred embodiment said amino acid sequence is derived from, homologous to or contiguous with amino acids 37-56, 46-65, 55-74, 109-128 and/or 127-146 inclusive or derivatives thereof of Hev b 5.

More preferably, said amino acid sequence is derived from, homologous to or contiguous with amino acids 46-65 and/or 109-128, and most preferably amino acids 51-60, inclusive or derivatives thereof of Hev b 5.

In another aspect said amino acid sequence comprises a sequence of at least 5 amino acids derived from one or more of the following amino acid sequences:

ASEQETADATPEKEEPTAAP <400> 6
TPEKEEPTAAPAEPEAPAPE <400> 7
APAEPEAPAPETEKAEEVEK <400> 8
TKETETEAPAAPAEGEKPAE <400> 14
AEEEKPITEAAETATTEVPV <400> 16

According to this aspect, said amino acid sequence preferably comprises a sequence of at least 5 amino acids derived from one or more of <400>7 or <400>14.

Still more preferably, said amino acid sequence comprises a sequence of at least 5 amino acids derived from <400>7.

Most preferably, said amino acid sequence comprises a sequence of at least 5 amino acids derived from the following amino acid sequence:

EPTAAPAEPE <400> 42.

The peptides of the present invention may be prepared by recombinant or chemical synthetic means. According to a preferred aspect of the present invention, there is provided a recombinant peptide which is preferentially immunologically reactive with T cells from individuals with latex hypersensitivity, which is expressed by the expression of a host cell transformed with a vector coding for the peptide sequence of the present invention. The peptide may be fused to another peptide, polypeptide or protein. Alternatively, the peptide may be prepared by chemical synthetic techniques, such as by the Merrifield solid phase synthesis procedure. Furthermore, although synthetic peptides of the formula given above represent a preferred embodiment, the present invention also extends to biologically pure preparations of the naturally occurring peptides or fragments thereof. By “biologically pure” is meant a preparation comprising at least about 60%, preferably at least about 70%, or preferably at least about 80% and still more preferably at least about 90% or greater as determined by weight, activity or other suitable means.

Still another aspect of the present invention is directed to antibodies to Hev b 5 including catalytic antibodies or derivatives, homologues, analogues, mutants, chemical equivalents or mimetics of said antibodies. Such antibodies may be monoclonal or polyclonal and may be selected from naturally occurring antibodies to Hev b 5 or may be specifically raised to Hev b 5. In the case of the latter, Hev b 5 may first need to be associated with a carrier molecule. The antibodies and/or recombinant Hev b 5 of the present invention are particularly useful as therapeutic or diagnostic agents. Alternatively, fragments of antibodies may be used such as Fab fragments or Fab′₂ fragments. Furthermore, the present invention extends to recombinant and synthetic antibodies and to antibody hybrids. A “synthetic antibody” is considered herein to include fragments and hybrids of antibodies. Hev b 5 can also be used to screen for naturally occurring antibodies to Hev b 5.

Both polyclonal and monoclonal antibodies are obtainable by immunization with Hev b 5 or derivative, homologue, analogue, mutant, chemical equivalent or mimetic thereof and either type is utilizable for immunoassays. The methods of obtaining both types of sera are well known in the art. Polyclonal sera are less preferred but are relatively easily prepared by injection of a suitable laboratory animal with an effective amount of Hev b 5, or antigenic parts thereof, collecting serum from the animal, and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example Douillard and Hoffman, Basic Facts about Hybridomas, in Compendium of Immunology Vol II, ed. by Schwartz, 1981; Kohler and Milstein, Nature 256: 495499, 1975; European Journal of Immunology 6: 511-519, 1976).

Preferably, the antibody of the present invention specifically binds Hev b 5 or derivative, homologue, analogue, mutant, chemical equivalent or mimetic thereof. By “specifically binds” is meant high avidity and/or high affinity binding of an antibody to a specific antigen. Antibody binding to its epitope on this specific antigen is stronger than binding of the same antibody to any other epitope, particularly those that may be present in molecules in association with, or in the same sample, as the specific antigen of interest. Antibodies that bind specifically to a polypeptide of interest may be capable of binding other polypeptides at a weak, yet detectable, level (e.g., 10% or less of the binding shown to the polypeptide of interest). Such weak binding, or background binding, is readily discernible from the specific antibody binding to the polypeptide of interest, e.g. by use of appropriate controls.

In a preferred embodiment the subject antibody is 1C10, 6F6, 3G3 or 6A10 and, more particularly, 1C10, 6F6 or 3G3 or derivative, homologue, analogue, chemical equivalent, mutant or mimetic thereof.

Hybridoma cell lines secreting 1C10, 6F6, 3G3 and 6A10 were deposited with ECACC on 19 Sep. 2000.

The development of monoclonal antibodies to Hev b 5 now permits the identification, isolation and synthesis of Hev b 5 B cell epitopes and the present invention should accordingly be understood to extend to Hev b 5 B cell epitopes and to derivatives, homologues, analogues, mutants, chemical equivalents or mimetics thereof.

In one preferred embodiment there is provided an isolated peptide of the formula:
X₁X₂X₃
wherein
X₁ and X₃ may be the same or different and each is an amino acid sequence comprising from 0 to 40 naturally or non-naturally occurring amino acid residues;
X₂ is an amino acid sequence derived from, or homologous to Hev b 5
   and wherein said peptide molecule is capable of interacting with antibody from subjects having a condition characterised by an aberrant, unwanted or otherwise inappropriate immune response to Hev b 5 or a derivative, homologue, analogue, mutant, chemical equivalent or mimetic of said peptide.

Preferably, said condition is latex hypersensitivity and said antibodies are of the IgE isotype.

More particularly the present invention provides an isolated peptide of the formula:
X₁X₂X₃
wherein
X₁ and X₃ may be the same or different and each is an amino acid sequence comprising from 0 to 40 naturally or non-naturally occurring amino acid residues;
X₂ is any amino acid sequence derived from, or homologous to Hev b 5
   and wherein said peptide molecule is capable of interacting with antibodies 1C10, 6F6, 3G3 or 6A10 or a derivative, homologue, analogue, mutant, chemical equivalent or mimetic of said peptide.

Reference to “Hev b 5”, and “peptide” should be understood to have the same meaning as hereinbefore defined. Similarly, reference to a condition involving an “aberrant, unwanted or otherwise inappropriate” immune response and “latex hypersensitivity” should also be understood to have the same meaning as hereinbefore provided. Further, reference to “derivatives, homologues, analogues, chemical equivalents and mimetics of the subject peptide or antibody also has the same meaning as provided earlier. The peptides encompassed by this aspect of the present invention may also undergo modification as hereinbefore detailed. Such modification is particularly useful for generating mutant peptides which are useful for a prophylactic and/or therapeutic treatment of individuals suffering from or predisposed to a condition characterised by an aberrant, unwanted or otherwise inappropriate immune response to Hev b 5. For example, it may be particularly useful to generate a mutant peptide comprising T cell epitopic regions but which peptides lack B cell epitopes capable of interacting with IgE. Such peptides may be generated by synthesising peptides comprising only T cell epitopes or by mutating naturally occurring molecules such that the T cell epitopes remain functional while the B cell epitopes are altered to prevent antibody binding. It should also be understood that the subject “amino acid sequence” may correspond to the sequence of either a continuous or discontinuous epitope of Hev b 5.

The present invention should therefore be understood to encompass peptides that comprise at least one B or T cell epitope of Hev b 5 in conjunction with other amino acids (which may or may not be naturally occurring as amino acid analogues) or other chemical species. In a preferred aspect of the invention such peptides may comprise one or more epitopes of Hev b 5, which epitopes may be T or B cell epitopes. Peptides with one or more epitopes of Hev b 5 are desirable for increased therapeutic effectiveness.

In another aspect, the present invention provides a nucleic acid molecule comprising a sequence of nucleotides encoding or complementary to a sequence encoding the peptides as hereinbefore defined or a derivative, homologue or analogue thereof. It should be understood that reference to “peptides” includes reference to peptides comprising one or more T cell epitopes, one or more B cell epitopes or a combination of B and T cell epitopes A nucleic acid molecule encoding the subject peptide is preferably a sequence of deoxyribonucleic acids such as cDNA or a genomic sequence. A genomic sequence may comprise exons and introns. A genomic sequence may also include a promoter region or other regulatory regions.

The nucleic acid molecule may be ligated to an expression vector capable of expression in a prokaryotic cell (eg. E. coli) or a eukaryotic cell (eg. yeast cells, fungal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector. The latter embodiment facilitates production of recombinant forms of the subject peptide which forms are encompassed by the present invention.

Such nucleic acids may be useful for recombinant production of T cell epitopes of Hev b 5 or proteins comprising them by insertion into an appropriate vector and transfection into a suitable cell line. Such expression vectors and host cell lines also form an aspect of the invention.

In producing peptides by recombinant techniques, host cells transformed with a nucleic acid having a sequence encoding a peptide according to the invention or a functional equivalent of the nucleic acid sequence are cultured in a medium suitable for the particular cells concerned. Peptides can then be purified from cell culture medium, the host cells or both using techniques well known in the art such as ion exchange chromatography, gel filtration chromatography, ultrafiltration, electrophoresis or immunopurification with antibodies specific for the peptide.

Nucleic acids encoding Hev b 5 or peptides comprising T and/or B cell epitopes of Hev b 5 may be expressed in bacterial cells such as E. coli, insect cells, yeast or mammalian cells such as Chinese hamster ovary cells (CHO). Suitable expression vectors, promoters, enhancers and other expression control elements are referred to in Sambruck et al (1989). Other suitable expression vectors, promoters, enhancers and other expression elements are well known to those skilled in the art. Examples of suitable expression vectors in yeast include Yep Sec 1 (Balderi et al., 1987); pMFa (Kurjan and Herskowitz, 1982); JRY88 (Schultz et al., 1987) and pYES2 (Invitrogen Corporation, San Diego, Calif.). These vectors are freely available as are baculovirus and mammalian expression systems. For example, a baculovirus system is commercially available (ParMingen, San Diego, Calif.) for expression in insect cells while the pMsg vector is commercially available (Pharmacia, Piscataway, N.J.) for expression in mammalian cells.

For expression in E. coli suitable expression vectors include among others, pTrc (Amann et al, 1988) pGex (Amrad Corporation, Melbourne, Australia); pMal (N.E. Biolabs. Beverley, MA); pRit5 (Pharmacia, Piscataway, N.J.); pEt-11d (Novagen, Maddison, Wis.) (Jameel et al 1990) and pSem (Knapp et al., 1990). The use of pTRC, and pEt-11d, for example, will lead to the expression of unfused protein. The use of pMa1, pRit5, pSem and pGex will lead to the expression of allergen fused to maltose E binding protein (pMal), protein A (pRit5), truncated-galactosidase (PSEM) or glutathione S-transferase (pGex). When a T cell epitope of Hev b 5 or a peptide comprising it is expressed as a fusion protein, it is particularly advantageous to introduce an enzymatic cleavage site at the fusion junction between the carrier protein and the peptide concerned. The peptide of the invention may then be recovered from the fusion protein through enzymatic cleavage at the enzymatic site and biochemical purification using conventional techniques for purification of proteins and peptides. Examples of enzymatic cleavage sites include those for blood clotting factor Xa or thrombin for which the appropriate enzymes and protocols for cleavage are commercially available. The different vectors also have different promoter regions allowing constitutive or inducible expression or temperature induction. It may additionally be appropriate to express recombinant peptides in different E. coli hosts that have an altered capacity to degrade recombinantly expressed proteins. Alternatively, it may be advantageous to alter the nucleic acid sequence to use codons preferentially utilised by E. coli, where such nucleic acid alteration would not effect the amino acid sequence of the expressed proteins.

Host cells can be transformed to express the nucleic acids of the invention using conventional techniques such as calcium phosphate or calcium chloride co-precipitation, DEAE-dextran-mediated transfection or electroporation. Suitable methods for transforming the host cells may be found in Sambruck et al (1989), and other laboratory texts. The nucleic acid sequence of the invention may also be chemically synthesised using standard techniques.

In addition to recombinant production of peptides according to the invention, the nucleic acids may be utilised as probes for experimental or purification purposes.

The identification of both B and T cell epitopic regions facilitates the identification and/or rational design of a range of mutant peptide molecules. As detailed hereinbefore, these mutant peptides may comprise one or more mutated T cell epitopes. However, in accordance with the antibody/B cell epitope related aspect of the present invention, there is provided scope for the generation of mutant peptides comprising mutated B cell epitopes or combinations of intact versus mutated B and T cell epitopes. The applications of these molecules are described in more detail below but in a preferred embodiment relate to modulation of the Hev b 5 hypersensitivity immune response in terms of either a prophylactic or therapeutic treatment.

Identification and synthesis of the Hev b 5 T and B cell epitopes and generation of Hev b 5 antibodies as disclosed herein now facilitates the development of a range of diagnostic and prophylactic/therapeutic treatment protocols for use with respect to Hev b 5 related immune conditions. Also facilitated is the development of reagents for use therein. Accordingly, the present invention should be understood to extend to the use of the peptides and monoclonal antibodies or derivatives, homologues, analogues, mutants, chemical equivalents or mimetics thereof of the present invention in the therapeutic and/or prophylactic treatment of patients. Such methods of treatment include, but are not limited to:
(i) Administration of the subject peptides to a patient as a means of desensitising or inducing immunological tolerance to Hev b 5 or Hev b 5-like molecules. This may be achieved, for example, by inducing Hev b 5 directed Th2 anergy or apoptosis. Such an outcome may be achieved by any one of a number of techniques including the use of peptides which maintain T cell epitope reactivity but which either naturally or as a result of mutation are unable to undergo IgE binding. Alternatively, one may utilise desensitisation/treatment protocols which are based on the administration of specific concentrations of a given peptide in accordance with a specific regime in order to induce high or low dose tolerance. Such methodology may eliminate Hev b 5 hypersensitivity or it may reduce the severity of Hev b 5 hypersensitivity.
   Preferably such treatment regimes are capable of modifying the T cell response or both the B and T cell response of the individual concerned. As used herein, modification of the allergic response of the individual suffering from Hev b 5 hypersensitivity can be defined as inducing either non-responsiveness or diminution in symptoms to the Hev b 5 molecule as determined by standard clinical procedures (Varney et al., 1990). Diminution in the symptoms includes any reduction in an allergic response in an individual to Hev b 5 after a treatment regime has been completed. This diminution may be subjective or clinically determined, for example by using standard skin tests known in the art.
   Exposure of an individual to the peptides of the present invention, which peptides comprise at least one T cell epitope, may tolerise or anergise appropriate T cell subpopulations such that they become unresponsive to Hev b 5 and do not participate in stimulating an immune response upon such exposure. Preferably the peptides according to the invention will retain immunodominent T cell epitopes but possess abrogated IgE binding.
   Administration of a peptide of the invention may modify the lympokine secretion profile as compared with exposure to naturally occurring Hev b 5 allergen. This exposure may also influence T cell subpopulations which normally participate in the allergic response to migrate away from the site or sites of normal exposure to the allergen and towards the site or sites of therapeutic administration. This redistribution of T cell subpopulations may ameliorate or reduce the ability of an individual's immune system to stimulate the usual immune response at the site of normal exposure to the allergen, resulting in diminution of the allergic symptoms.
   Modification of the B cell response may be achieved, for example, via modulation of the cytokine profile produced by T cells, as detailed above. Specifically, decreasing T cell derived IL-4 and IL-13 production thereby decreasing IgE synthesis.
(ii) The peptides of the present invention may be used in the capacity of an adsorbent to remove Hev b 5 directed antibodies and/or T cells from a biological sample or from a patient.
(iii) The antibodies generated in accordance with the method of the present invention following humanisation or modification to produce Fabs, for example, may be used to abrogate or decrease Hev b 5 hypersensitivity via the administration to an individual of antibodies which bind Hev b 5 thereby competitively inhibiting binding of Hev b 5 to IgE coated mast cells, which latter binding would lead to mast cell degranulation and the onset of hypersensitivity symptoms. Although such a mechanism is unlikely to lead to long term desensitisation, it nevertheless provides a mechanism for preventing or reducing acute hypersensitivity symptom severity. This is likely to be of particular use to individuals who suffer from severe symptoms following certain types of exposure to Hev b 5, such as airway constriction.

Accordingly, in another aspect the present invention provides a method for the treatment and/or prophylaxis of a condition in a subject, which condition is characterised by the aberrant, unwanted or otherwise inappropriate immune response to Hev b 5, said method comprising administering to said subject an effective amount of a peptide and/or antibody as hereinbefore defined for a time and under conditions sufficient to remove or reduce the presence or function in said subject of T cells and/or antibodies directed to said Hev b 5.

Preferably said condition is latex hypersensitivity.

An “effective amount” means an amount necessary at least partly to attain the desired immune response, or to delay the onset or inhibit progression or halt altogether, the onset or progression of a particular condition being treated. The amount varies depending upon the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials.

The subject of the treatment or prophylaxis is generally a mammal such as but not limited to human, primate, livestock animal (e.g. sheep, cow, horse, donkey, pig), companion animal (e.g. dog, cat), laboratory test animal (e.g. mouse, rabbit, rat, guinea pig, hamster), captive wild animal (e.g. fox, deer). Preferably the mammal is a human or primate. Most preferably the mammal is a human. Although the present invention is exemplified using a murine model, this is not intended as a limitation on the application of the present invention to other species, in particular, human.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

Administration of the peptide and/or antibody of the present invention (herein referred to as “agent”) in the form of a pharmaceutical composition, may be performed by any convenient means. The agent of the pharmaceutical composition is contemplated to exhibit therapeutic activity when administered in an amount which depends on the particular case. The variation depends, for example, on the human or animal and the agent chosen. A broad range of doses may be applicable. Considering a patient, for example, from about 0.1 mg to about 1 mg of an agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The agent may be administered in a convenient manner such as by the oral, intravenous (where water soluble), intraperitoneal, intramuscular, subcutaneous, intradermal, intranasal, sublingual or suppository routes or implanting (e.g. using slow release molecules). The agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

Another aspect of the present invention contemplates the use of an agent as hereinbefore defined in the manufacture of a medicament for the treatment of a condition in a mammal, which condition is characterised by an aberrant, unwanted or otherwise inappropriate immune response to Hev b 5.

Preferably said condition is latex hypersensitivity.

In yet another further aspect, the present invention contemplates a pharmaceutical composition comprising an agent as hereinbefore defined and one or more pharmaceutically acceptable carriers and/or diluents. Said agents are referred to as the active ingredients.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antiftngal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding a modulatory agent. The vector may, for example, be a viral vector.

Yet another aspect of the present invention relates to agents, as hereinbefore defined, when used in the method of the present invention.

In yet another aspect, the present invention should be understood to extend to the use of the peptides and/or antibodies of the present invention in diagnostic applications. Said diagnostic applications include, but are not limited to:
(i) To measure the reactivity of a subject's cells to Hev b 5. This is of use, for example, with respect to the diagnosis and/or monitoring of conditions characterised by an aberrant, unwanted or otherwise inappropriate immune response to Hev b 5. The peptides may be added into solution or bound to a solid support together with cells derived from peripheral blood or from tissue biopsies either unfractionated, fractionated or derived as a continuous cell line. Reactivity to the subject peptide may then be measured by standard proliferation assays such as incorporation of H³-thymidine, standard cytotoxic assays such as release of marker radioactivity from target cells, measurement of expressed or secreted molecules such as surface markers, cytokines or other standard assays of cellular activity which are well known in the art.
(ii) The use of T cell epitope comprising peptides together with a T cell proliferation assay which utilises a T cell sample derived from the subject will facilitate, for example, the identification of a T cell responsive population.
   B cell epitope comprising peptides can be utilised to screen for the presence of antibody at the qualitative and/or quantitative levels.
(iii) The antibodies generated in accordance with the present invention may be utilised as a diagnostic tool for screening for the presence of molecules comprising Hev b 5 cell epitopes in a sample. Said sample may be a biological sample, such as where it is suspected that an individual may have ingested Hev b 5 comprising material. For example, detection of Hev b 5 homologues in patients suffering from suspected fruit hypersensitivity. In another example, it may be desirable to test non-biological samples, such as those suspected of inducing hypersensitivity responses in some individuals who contact these samples, for the presence of Hev b 5. For example, it may be desirable to test rubber gloves in order to assess their qualitative or quantitative Hev b 5 composition.
   The methods which can be utilised to screen for Hev b 5 on the basis of B cell epitope detection using antibody molecules are well known to those skilled in the art and include radio-allergosorbent test (RAST), paper radio immunoabsorbent test (PRIST), enzyme linked immunoabsorbent assay (ELISA), radio-immunoassay (RIA), immunoradiometric assay (IRMA), luminescence immunoassay (LIA), histamine release assays and IgE immunoblots.
   As detailed above, techniques for the assays contemplated herein are known in the art and include, for example, sandwich assays, ELISA and flow cytometry. It is within the scope of this invention to include any second antibodies (monoclonal, polyclonal or fragments of antibodies) directed to the first mentioned antibodies discussed above. Both the first and second antibodies may be used in detection assays or a first antibody may be used with a commercially available anti-immunoglobulin antibody. An antibody as contemplated herein includes any antibody specific to any region of Hev b 5.
   To the extent that antibody based methods of diagnosis are used, the presence of Hev b 5 may be determined in a number of ways such as by Western blotting, ELISA or flow cytometry procedures. These, of course, include both single-site and two-site or “sandwich” assays of the non-competitive types, as well as in the traditional competitive binding assays. These assays also include direct binding of a labelled antibody to a target.
   Sandwich assays are among the most useful and commonly used assays and are favoured for use in the present invention. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent.
   In the typical forward sandwich assay, a first antibody having specificity for the Hev b 5 or antigenic parts thereof, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes) and under suitable conditions (e.g. 25° C.) to allow binding of any subunit present in the antibody. Following the incubation period, the antibody subunit solid phase is washed and dried and incubated with a second antibody specific for a portion of the hapten. The second antibody is linked to a reporter molecule which is used to indicate the binding of the second antibody to the hapten.
   An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.
   By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.
   In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. “Reporter molecule” also extends to use of cell agglutination or inhibition of agglutination such as red blood cells on latex beads, and the like.
   Alternately, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength the fluorescence observed indicates the presence of the hapten of interest. Immunofluorescene and EIA techniques are both very well established in the art and are particularly preferred for the present method. However, other reporter molecules, such as radioisotope, chemiluminescent or bioluminescent molecules, may also be employed.
(iv) Anti-Hev b 5 antibodies may also be utilised as a means of purifying Hev b 5 or the T or B cell epitope comprising peptides thereof which have been made by recombinant means or derived from any other source, such as natural products. Such purification techniques may be applied to isolating Hev b 5 proteins from culture supernatant fluid or from natural products, for example.

Methods of detecting Hev b 5 may be utilised, for example, to qualitatively or quantitatively detect Hev b 5 levels. However, these methods may also be utilised to screen for mutations or polymorphisms in Hev b 5 which mutations may result in, for example, loss of T cell and/or B cell reactivity to Hev b 5. These methods may be utilised for the purpose of screening for peptide molecules suitable for use in therapeutically or prophylactically treating an individual suffering from Hev b 5 related hypersensitivity.

Accordingly, yet another aspect of the present invention is directed to a method of diagnosing or monitoring a condition in a mammal, which condition is characterised by an aberrant, unwanted or inappropriate response to Hev b 5, said method comprising screening for Hev b 5 reactive T cells and/or antibodies utilising the peptides hereinbefore defined.

Preferably said condition is latex hypersensitivity.

Still another aspect of the present invention is directed to a method of qualitatively and/or quantitatively detecting Hev b 5, or peptides thereof, in a sample said method comprising screening for said Hev b 5 or peptides thereof utilising an antibody hereinbefore defined.

In another embodiment the present invention provides diagnostic kits for use in the diagnostic methodology hereinbefore defined.

The present invention will now be further described with reference to the following non-limiting Examples.

### EXAMPLE 1

### Identification of Human T Cell Epitopes of Hev b 5

(i) Methods

Subjects

Latex allergic HCW were recruited from the Alfred Hospital Allergy Clinic (with informed consent following approval by Alfred Hospital Ethics Committee). All subjects had severe clinical symptoms of IgE mediated latex hypersensitivity and a grade 3-4/4 score of latex-specific serum IgE (Table 3) measured using the Kallestad Allercoat™ EAST system (Sanofi-Pasteur Diagnostics, USA). The latex EAST score in non-allergic individuals is 0/4 and <0.18 Allercoat™ EAST Units (AEU/ml).

Antigens

Low-ammoniated latex: Low ammoniated latex (LAL; Ansell, Australia) was centrifuged at 20,000 rpm for 20 minutes at room temperature. The middle clear layer was collected, dialysed against phosphate buffered saline (PBS) overnight at 4° C., filter sterilised and the protein concentration determined using the BCA protein assay kit (Pierce, USA).

Hev b 5: A construct comprising the Hev b 5 encoding cDNA in the pMAL/c-2 vector was generated as part of a maltose binding protein (MBP) fusion protein (rHev b 5/MBP) as described previously (Slater et al., 1996). The rHEV b 5/MBP was purified from the crude bacterial protein extract by affinity chromatography on an amylose column (New England Biolabs, USA) according to the manufacturer's instructions. As a control protein, MBP fused to the LacZa protein was produced by expressing the pMAL/c-2 vector alone as described previously (Slater et al., 1996).

Tetanus toxoid (TT) and Phytohaemagglutinin (PHA): These control antigens were purchased from Sigma, USA.

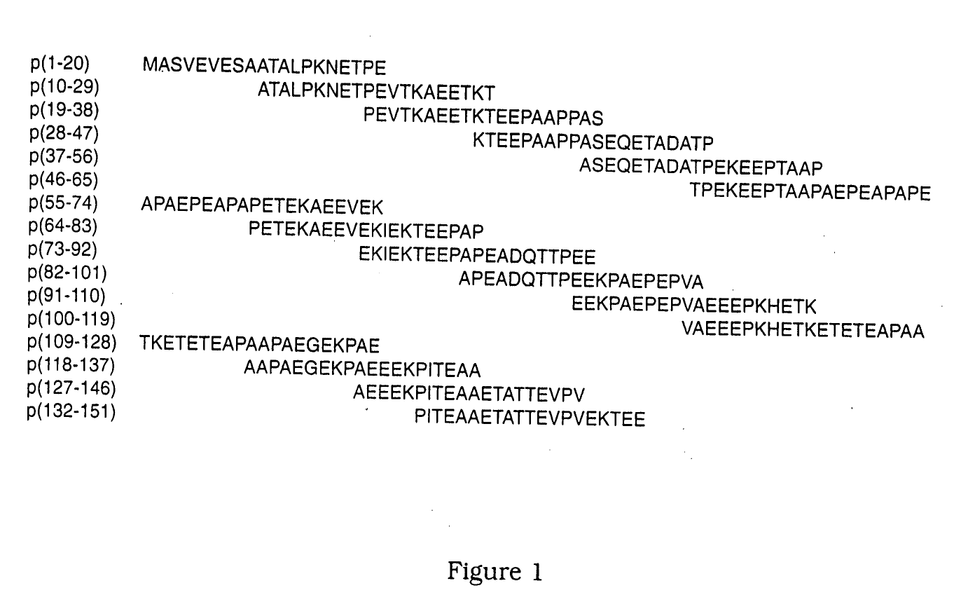

Peptides: Synthetic peptides (20-mers overlapping by 11 or, for the two N-terminal peptides, 15 residues) spanning the entire length of the Hev b 5 molecule were purchased from Chiron Technologies (Clayton, Australia; FIG. 1). The purity of the peptides, as determined by high-performance liquid chromatography, was greater than 90% Peptides were dissolved in sterile PBS (Sigma, USA) to achieve a stock concentration of 1 mg/ml.

Immunoblotting

Patient serum IgE reactivity to latex allergens was analysed by dot immunoblots. LAL (25 μg), rHev b 5/MBP (7 μg) and MBP (4.5 μg) were applied side by side on a nitrocellulose membrane (Schleichner and Schuell, Germany) in a 5 μl volume and allowed to soak in. The amount of MBP was estimated to be equivalent to that in the rHev b 5/MBP aliquot. The membrane was then blocked, incubated in patient sera and IgE binding detected according to our established protocols (Suphioglu et al., 1993). Briefly, the blots were blocked in 10% w/v skim milk powder in PBS and washed once in PBS containing 0.2% v/v Tween 20 and twice in PBS alone. Blots were then incubated in patients' sera (diluted 1:4 in PBS/0.5% BSA) overnight at room temperature with shaking before washing as described above. Finally, IgE antibody binding was detected by incubation in rabbit anti-human IgE (Dako, Denmark) followed by horse radish peroxidase-labelled anti-rabbit antibodies (Promega, USA), washing in between, and developing in the substrate 4-chloro-1-napthol (Sigma) after the final wash.

Generation of Latex Specific T Cell Lines

Latex specific T cell lines were isolated using our well established methods for the generation of allergen specific oligoclonal T cell populations (O'Hehir et al., 1987). An initial primary culture was performed for each patient to determine the optimal concentration of LAL for stimulation. For this, peripheral blood mononuclear cells (PBMC) separated from heparinised venous blood by density centrifugation (2×10⁵/well) were stimulated with LAL over a concentration range 0.03 to 100 μg/ml, with control cultures of PBMC alone or stimulated with tetanus toxoid. All cultures were maintained in complete medium (RPMI-1640, Gibco Life Technologies supplemented with 2 mmol/L L-glutamine, 100 IU/ml penicillin-streptomycin and 5% screened, heat inactivated human AB serum [Sigma]). Optimal responses were observed at 10 and 30 μg/ml LAL in all cases. Therefore, to generate T cell lines, PBMC (2.5×10⁶/well) were stimulated for 1 week with LAL at a concentration of 20 μg/ml in 24-well tissue culture plates (Costar, USA). After 7 days, lymphoblasts (1×10⁶/well) were restimulated for one week with LAL at a concentration of 20 μg/ml in the presence of an equal number of irradiated (3000 rad) autologous PBMC as antigen presenting cells (APC). On days 2 and 4, cultures were supplemented with Lymphocult-T (5% v/v; Biotest Folex, Germany) and fresh medium. At the end of the second week, lymphoblasts were restimulated for one week with rhev b 5/MBP at a concentration of 20 μg/ml as described above. Oligoclonal T cells were recovered, washed and tested in proliferation and cytokine assays (as described below) either fresh or after cryopreservation in liquid nitrogen. We have previously shown that CD4⁺ T cells are preferentially expanded in these cultures.

Oligoclonal T Cell Proliferation Assays

Oligoclonal T cell blasts (5×10⁴) were incubated in 96-well round bottom plates (Linbro, ICN Biomedicals, USA) in triplicate with equal numbers of irradiated APC in the presence of LAL at concentrations ranging from 0.03-100 μg/ml, rHev b 5/MBP (10 and 20 μg/ml), MBP (10 and 20 μg/ml), overlapping peptides of the Hev b 5 sequence (10 and 30 μg/ml), Lymphocult-T (10 IU/ml) or PHA (3 μg/ml). Cultures of T cells and APC in the absence of antigen, T cells alone and APC alone in the absence of antigen were included as controls. After 72 hours cultures were pulsed with 1 μCi of ³H-thymidine (³HTdR; Du Pont, NEN) and harvested 12-16 hours later. Proliferation as correlated with ³HTdR incorporation was measured by liquid scintillation spectroscopy. Results are expressed as mean counts per minute (cpm) for triplicate cultures with standard deviation (SD; ±20% for all experiments).

Mitogenicity and Toxicity Assays

Mitogenicity of latex antigens was excluded by testing the ability of LAL, rHev b 5/mBP and Hev b 5 peptides to stimulate a house dust mite specific three-week T cell line. No reactivity to latex allergens was observed (data not shown). Toxicity was assessed by coculturing the same antigens with house dust mite specific T cells in the presence of Lymphocult-T at 10 IU/ml. Latex antigens failed to inhibit the Lymphocult-T induced proliferation even at the highest concentration (data not shown) excluding toxicity.

Production of IL-5 and IFNγ by Latex Specific Oligoclonal T Cells

The secretion of IL-5 and IFNγ by oligoclonal latex specific T cells was determined by ELISA on culture supernatants. Representative stimulatory and non-stimulatory peptides were selected and T cells cultured in triplicate with these peptides. At 48 hours 50 μl of supernatant was harvested from the cultures and IL-5 and IFNγ were assayed by ELISA using paired cytokine antibodies (from Pharmingen Becton Dickinson, USA and Endogen, USA respectively). For this, opaque flat bottom ELISA plates (Nalgene, Nunc International) were coated with 30 μl capture antibody at 2 μg/ml in 0.1 M NaHCO₃ pH 8.2 coating buffer overnight at 4° C. After washing three times with PBS/0.5% Tween the wells were blocked with 3% BSA/PBS at room temperature for 2 hours. The plates were washed and 30 μL pooled triplicate culture supernatants added in duplicate. After incubation overnight at 4° C. and washing, bound cytokine was revealed by incubation with 30 μl biotinylated detecting antibody (1 μg/ml for IL-5, 0.5 μg/ml for IFNγ) for 45 minutes at room temperature followed by streptavidin-biotinylated HRP (Amersham Pharmacia Biotech) at 1:2000 dilution for 30 minutes and enhanced chemiluminescence substrate (NEN Life Science). Light emission was read immediately on a Lumicount microplate Glow Luminometer (Packard Instrument Company). A standard curve was run each time using known concentrations of standard cytokine samples and the concentrations of IL-S and IFNγ in the culture supernatants calculated accordingly. The lower limit of detection of the IL-5 assay was 20 pg/ml and the lower limit of detection of the IFN-γ assay was 2 pg/ml.

(ii) Results

Serum IgE Response to Hev b 5

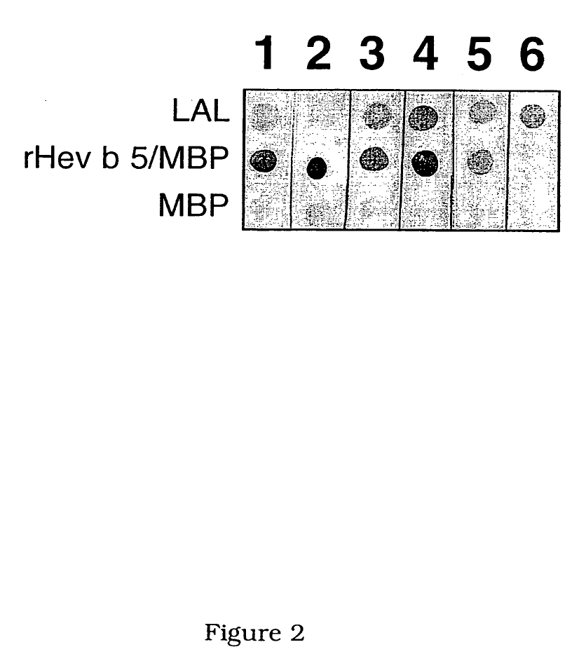

Sera from the latex allergic HCW were tested for IgE reactivity to latex allergens by dot immunoblotting using LAL and rHev b 5/MBP as antigens. MBP was included as a control to exclude the possibility that binding of patient sera to the fusion protein was due to anti-MBP antibodies. All patients except patient 2 demonstrated IgE reactivity to LAL (FIG. 2). Sera from patients 1, 2, 3, 4 and 5 showed IgE reactivity to rHev b 5/MBP which was markedly stronger than that to MBP alone indicating the presence of a B cell response to Hev b 5 in these patients. Patient 6 showed IgE reactivity to LAL but not to the rHev b 5/MBP fusion protein indicating that Hev b 5 was not a significant allergen in this case.

T Cell Responses to Latex Allergens

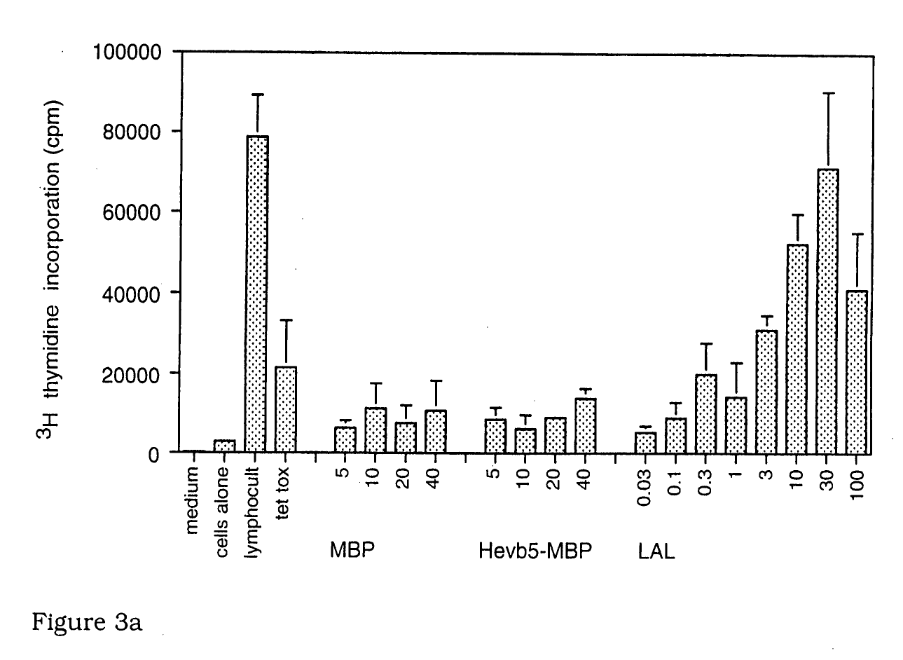

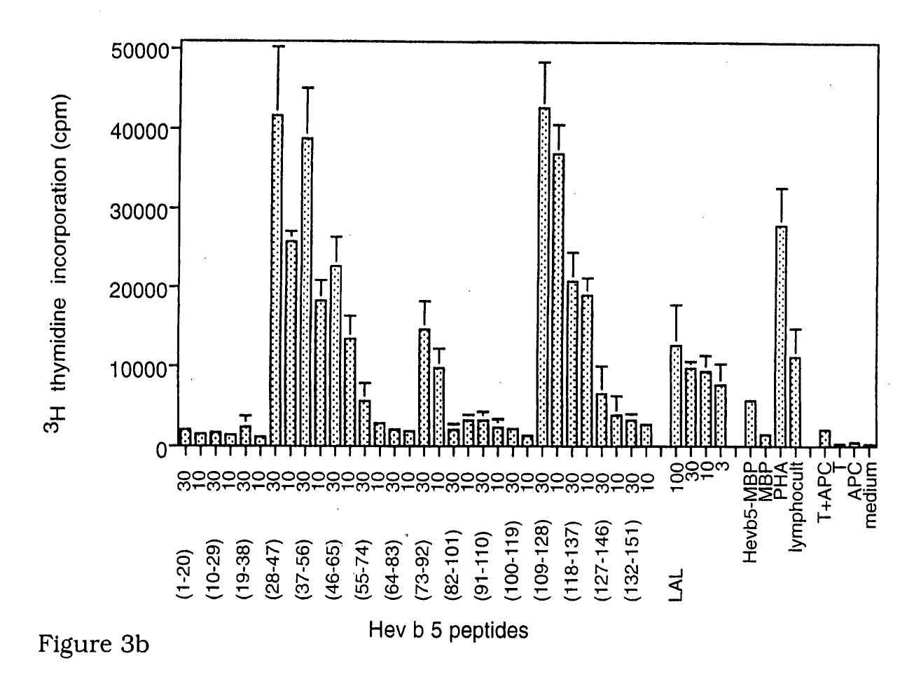

In preliminary primary cultures of PBMC, the optimal response to LAL was observed at 10 or 30 μg/ml in all cases. A representative polyclonal response is shown in FIG. 3, (A). The polyclonal responses to rHev b 5/MBP were considerably less than those to LAL. A similar response was observed to MBP alone indicating the presence of MBP-specific T cells in the peripheral blood. Therefore, to enrich for latex specific T cells while preventing the selective expansion of MBP-specific T cells, LAL at 20 μg/ml was used for primary and secondary stimulations. Finally, Hev b 5 specific T cells were enriched by incubating the LAL specific two-week T cell line with rHev b 5/MBP at 20 μg/ml in the third antigen stimulation. The three-week latex specific T cell lines from all six latex allergic donors responded to LAL (Table 4). The T cell lines from patients 1 and 2 were also tested for their proliferative responses to rHev b 5/MBP and MBP alone. In contrast to the polyclonal responses, the oligoclonal responses to rHev b 5/MBP were significantly higher than those of MBP alone, indicating a selective expansion of Hev b 5-specific T cells. In fact, MBP responses were only at background levels. Representative responses to LAL, rHev b 5/MBP and MBP are shown FIG. 3, (B).

Hev b 5 T Cell Epitope Mapping

The latex specific three-week T cell lines were tested for proliferative responses to the Hev b 5 peptides. T cell reactivity to one or more Hev b 5 peptides was identified in all five donors who demonstrated IgE binding to rHev b 5/MBP (Table 4), and a representative T cell proliferative response to the peptide set is shown (FIG. 3, B). Patient 6 failed to respond to any peptide. Hev b 5 (46-65) induced T cell proliferation in all five donors with IgE reactivity to rHev b 5/MBP. The stimulation indices observed for these responses were 5.9, 10.8, 5.0, 5.9 and 5.6. Additionally peptide Hev b 5 (109-128) stimulated T cells from three of the five donors.

Production of IL-5 and IFNγ by Oligoclonal Latex Specific T Cells

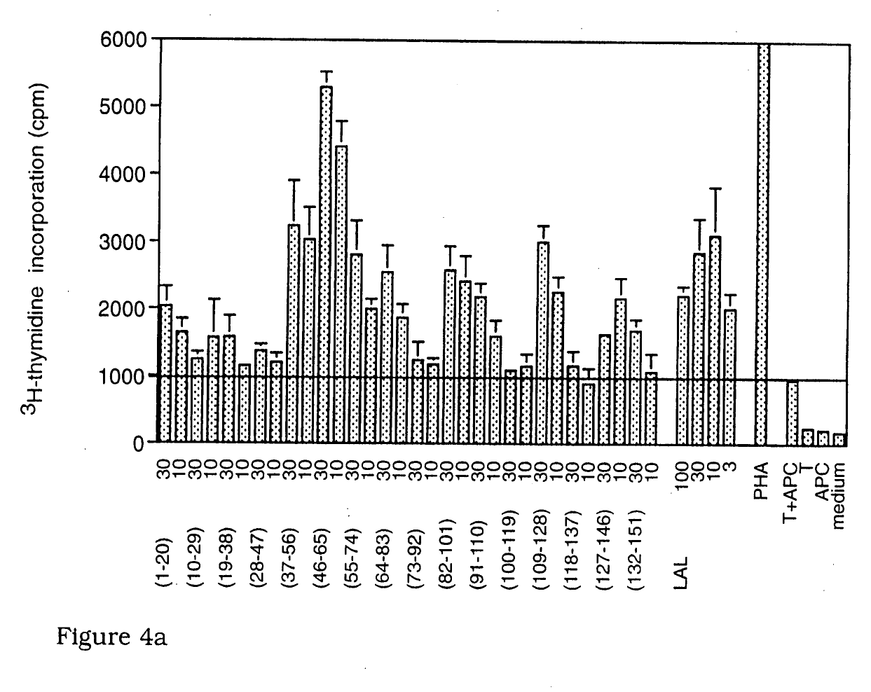

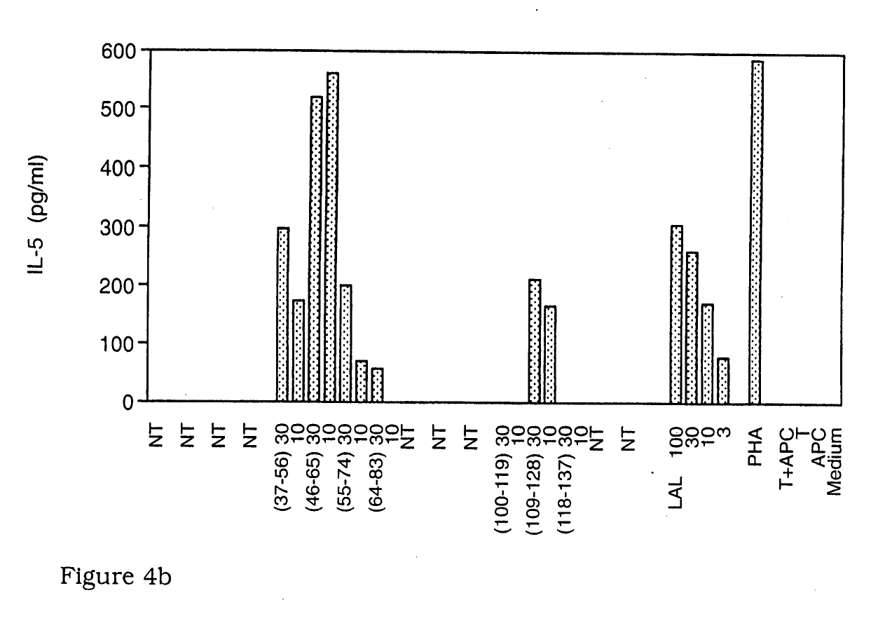

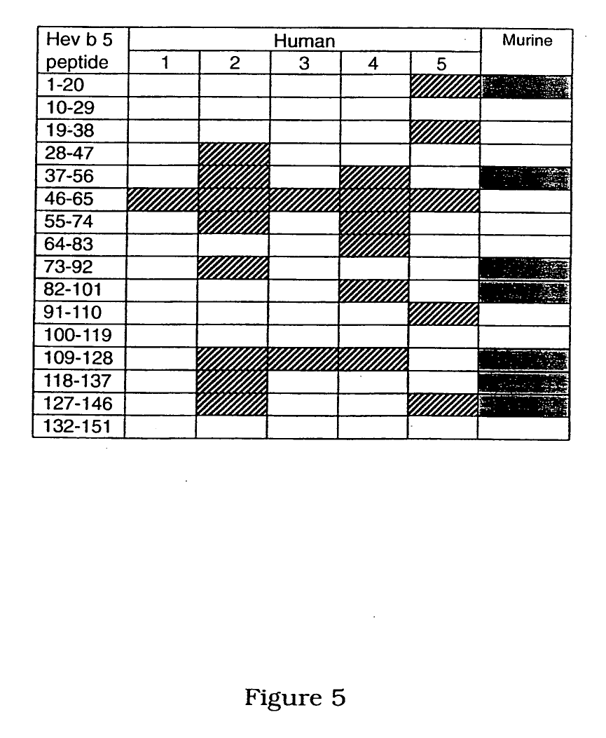

IL-5 was produced by the latex specific T cell line from patient 4 in response to stimulation with selected Hev b 5 peptides that caused T cell proliferation (FIG. 4). Peptides Hev b 5 (37-56), (45-65), (55-74), (64-83) and (109-128) induced T cell proliferation (stimulation indices greater than or equal to 2.5) and also secretion of IL-5 while peptide Hev b (100-119) failed to stimulate either T cell proliferation or cytokine secretion. In contrast secretion of IFNγ by T cells that proliferated in response to Hev b 5 peptides was minimal in all cases (≦100 pg/ml). See Table 5.

### EXAMPLE 2

### Generation & Characterisation of Hev b 5 Specific Monoclonal Antibodies

(i) Materials and Methods

Antigens

rHev b 5/MBP—The cDNA encoding Hev b 5 was expressed as a fusion protein with MBP in the pMAL/c-2 vector as described previously (Slater et al. 1996). The rHev b 5/MBP was purified by affinity chromatography on an amylose column (New England Biolabs, USA) according to the manufacturer's instructions. MBP encoding DNA fused to the LacZα protein was produced by expressing the pMAL/c-2 vector alone and was used as the negative control protein. The rHev b 5/4BP fusion protein digest was made by incubating 500 μg rHev b 5/MBP with 10U Factor Xa (Sigma. USA) and 0.03% SDS for 24 hours at 37° C.

Non Ammoniated Latex (NAL)—fresh sap was collected from commercial Hevea trees into a cooling vessel and snap frozen (Rubber Research Institute of Malaysia, Malaysia) before transport to our facility on dry ice. The raw latex was then thawed and the coagulum removed. The sap was then centrifuged for 1 hour at 50,000 g and the C-serum removed for protein estimation using the Bradford assay (Biorad, USA) with bovine gamma globulin (Biorad, USA) as standard. It was then mixed 50% v/v in glycerol and stored at −20° C.

Monoclonal Antibody Production

Monoclonal antibody production was carried out based on the method of Köhler and Milstein (Köhler et al., 1975) according to established protocols (Goding, 1996). Six to 8 week old female BALB/c mice were immunised intraperitoneally (IP) with 20 μg/injection of rHev b 5/MBP diluted in 200 μl of phosphate buffered saline (PBS). The first injection was given with an equal volume (200 μl) of Complete Freund's Adjuvant (Sigma, USA), and the second with 200 μl of Incomplete Freund's Adjuvant (Sigma, USA). The subsequent 4 immunisations were given in phosphate buffered saline (PBS) alone. The initial 2 immunisations were given at a 2 weekly interval; the remainder were given at monthly intervals, with 100 μl of whole blood being taken before the first immunisation and at 5 days following each immunisation (except for the first) to monitor the immune response. The blood was then centrifuged, the serum removed, and aliquotted at −20° C. for later analysis using enzyme linked immunosorbent assay (ELISA) as below. The final booster immunisation of 20 μg of rHev b 5/MBP in 200 μl PBS was given IP 3 days before the mouse was humanly killed and the spleen aseptically removed, macerated and then fused with 10⁷ murine myeloma cells (X-63.Ag8 cell line, generous gift of Professor Jim Goding). The fusion method used was with 50% PEG mw 1300-1600 (Sigma, USA), with gentle mixing at 37° C. for 4 minutes. The fusion was then diluted into 60 mls of selection medium: RPMI 1640 (Life Technologies, USA) supplemented with 20% fetal calf serum (FCS) (CSL. Australia), 10 mls of 50×HAT medium (Sigma, USA, 5 mls of Penicillin-Streptomycin-Glutamine (PSG) (Life Technologies, USA) and 10 mls of Oxalate-Pyruvate Insulin supplement (OPI) (Sigma, USA). This cell suspension was then plated out into 6 flat bottom 96 well tissue culture plates (100 μl/well) (Linbro, USA) and incubated at 37° C. with 7% CO₂. Further fresh medium (100 μl per well of RPMI with 10% FCS, HAT, PSG) was added 5 days following fusion. Seven days following fusion hybridomas were visible by eye in many wells and the first screening was performed. Supernatants were removed (100 μl per well) and transferred to microtitre trays. A duplicate ELISA was then performed for each plate against rHev b 5/MBP and MBP alone. Briefly, ELISA plates (Costar, USA) were coated overnight (O/N) with 50 μl per well of rHev b 5/MBP or MBP alone at 0.2 μg/ml in ELISA coating buffer pH 9.5 (2.52 g NaHCO₃+1.59 g Na₂CO₃ per 500 ml distilled H₂O). The plates were then washed 8 times in PBS with 0.5% Tween 20 (BDH, England) (PBST) and then blocked with 5% w/v skim milk powder (SMP) in PBST O/N at 4° C. before being washed again 8 times in PBST. Hybridoma supernatants were then incubated (50 μl/well) for 1 hour at 37° C., the plates then washed 8 times in PBST then 50 μl per well of horse-radish peroxidase (HRP) labelled sheep anti-mouse antibody (Silenius, USA) diluted 1:5000 in 1% bovine scnim albumin (BSA) PBST was added and incubated for 1 hour at 37° C. The plates were then washed 4 times with PBST and then 4 times with PBS alone before the addition of 50 μl/well of a solution of 50 mg O-phenylene diamine (OPD) (Sigma, USA) in 12.5 ml of phosphate citrate buffer with perborate (Sigma, USA). This was incubated for 10 minutes at 37° C. and the reaction stopped with 4M HCl. Colour development was then observed and the plate read at 490 mm. In all cases, one well of the ELISA plate had cell culture medium alone as a negative control and the terminal bleed of the fusion mouse at a 1:5000 dilution in 1% BSA PBST as positive control.

Positive clones were then transferred to 24 well plates (Costar, USA) in RPMI with 10% FCS, PSG and HT supplement (Sigma, USA) then expanded before cloning by limiting dilution in flat bottom 96 well plates. Limiting dilution cloning was performed in the presence of feeder cells (splenocytes from female BALB/c mice) suspended in RPMI 1640, 10% FCS, PSG, HAT and OPI. Positive clones were again screened by ELISA against rHev b 5/MBP, MBP at a concentration of 10 μg/ml in ELISA coating buffer pH 9.5.

Western Blotting

Extracts of NAL (35 μg/lane) and rHev b 5/MBP digest (10 μg/lane) were resolved on 12% SDS-PAGE (125V D for 90 minutes) with Benchmark pre-stained protein ladder (Life Technologies, USA) as molecular weight standards. Gels were then stained with Coomassie Brilliant Blue for total protein visualisation or electroblotted to nitrocellulose (NC) membranes (Schleicher and Schuell, Germany) for 45 minutes at 25V D. After transfer, NC membranes were then stained with 0.1% Ponceau S to ensure protein transfer and cut into strips. They were then blocked with 5% SMP in PBST for 1 hour at RT, washed twice in PBST and then incubated with hybridoma supernatants or mouse serum for 1 hour at RT. Blots were then washed 5 times in PBST and then incubated in sheep anti-mouse HRP diluted 1:10,000 in PBST. The blots were then washed 5 times in PBST and then incubated in enhanced chemiluminescence reagent (ECL) (NEN Life Sciences, USA) as per the manufacturer's instructions and luminescence detected on autoradiography film (Kodak, Australia).

(ii) Results

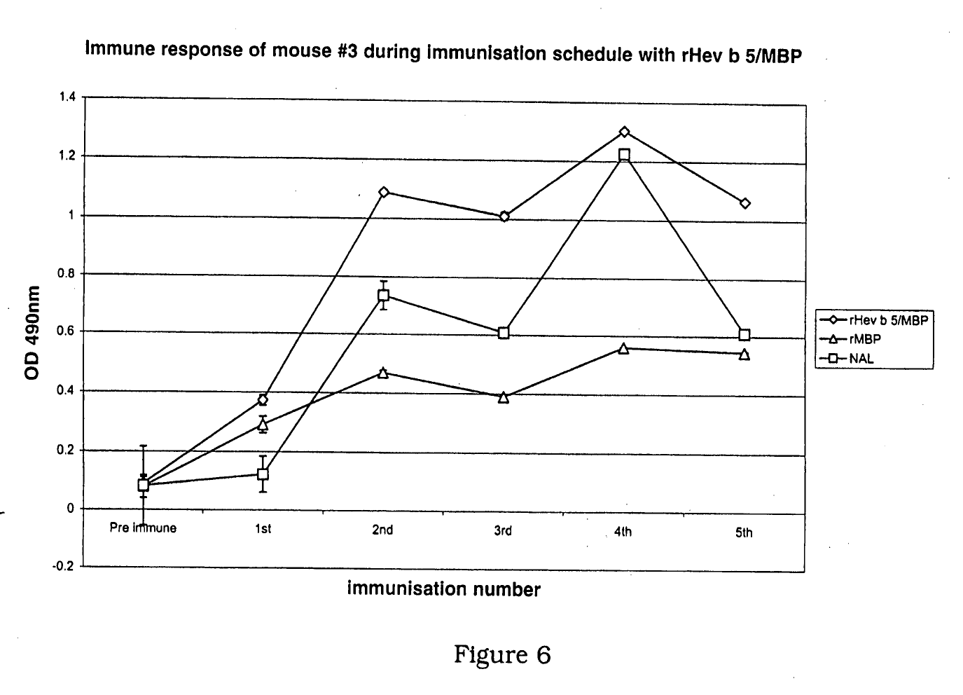

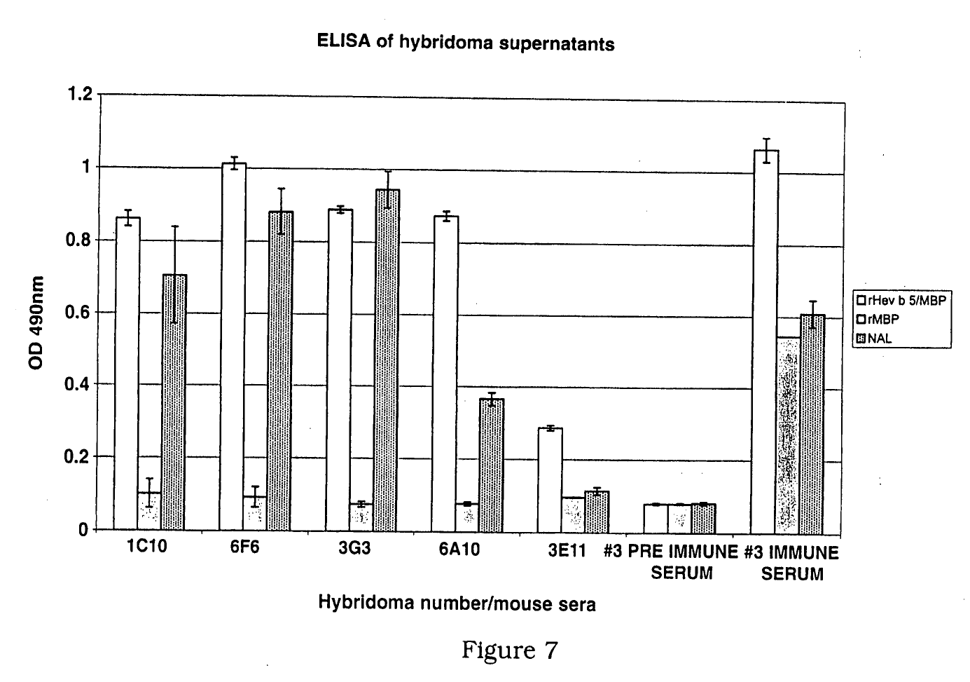

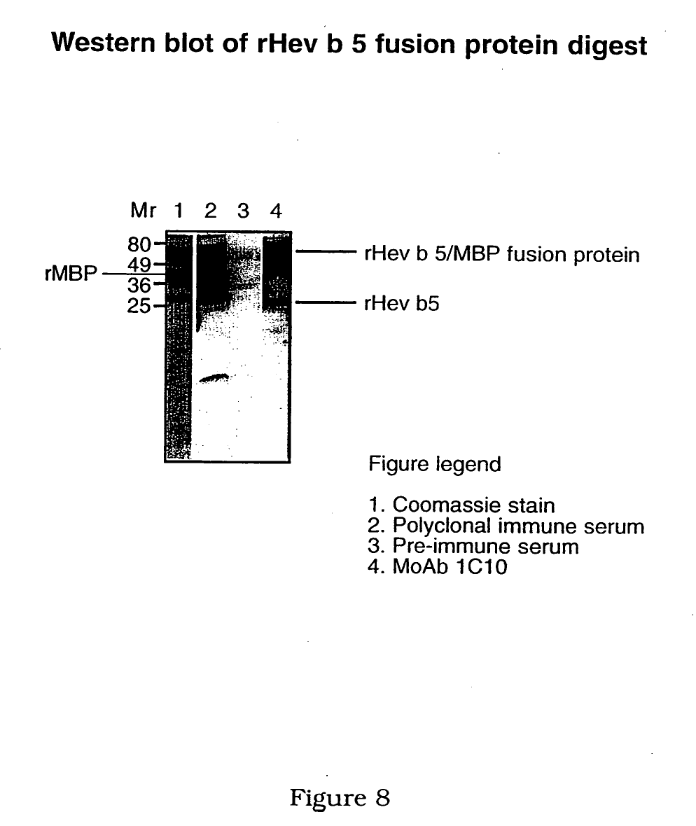

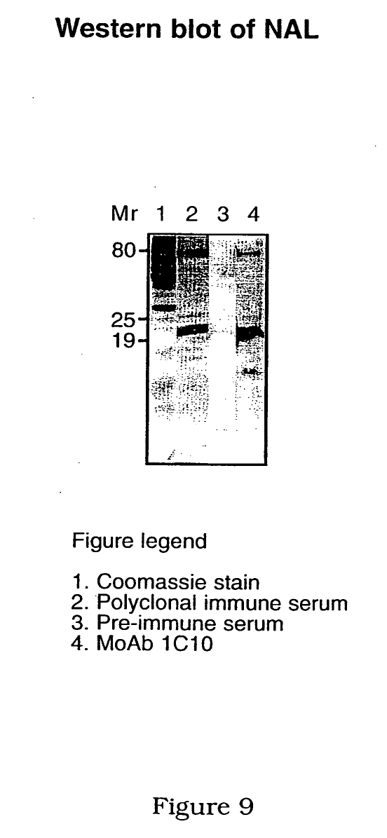

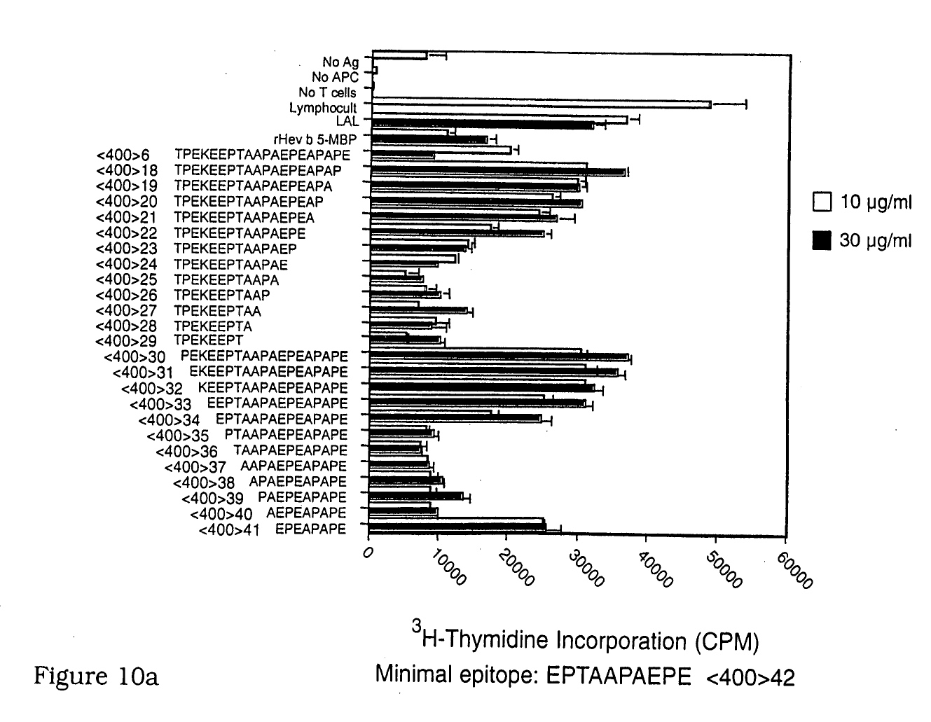

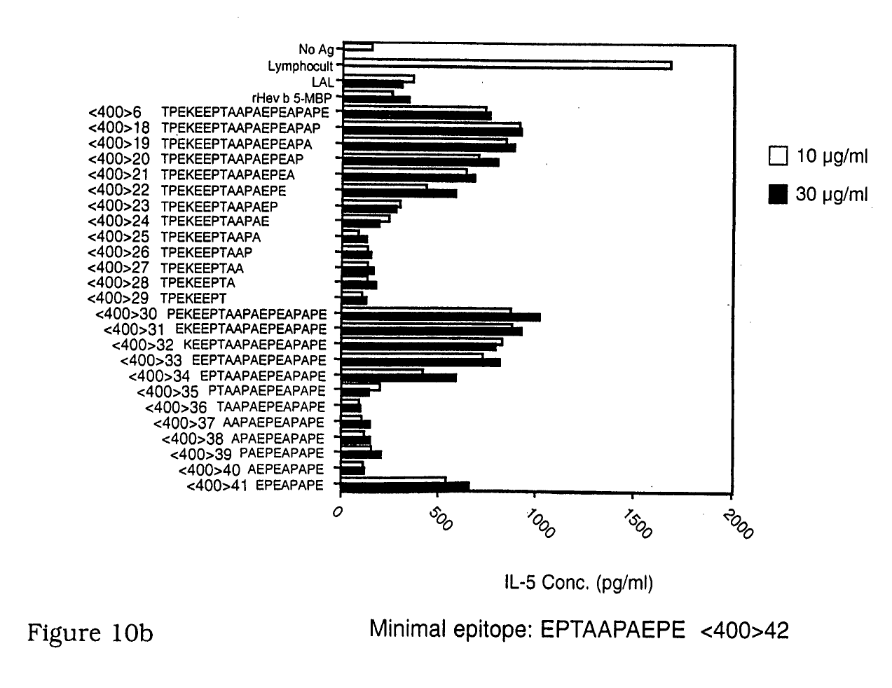

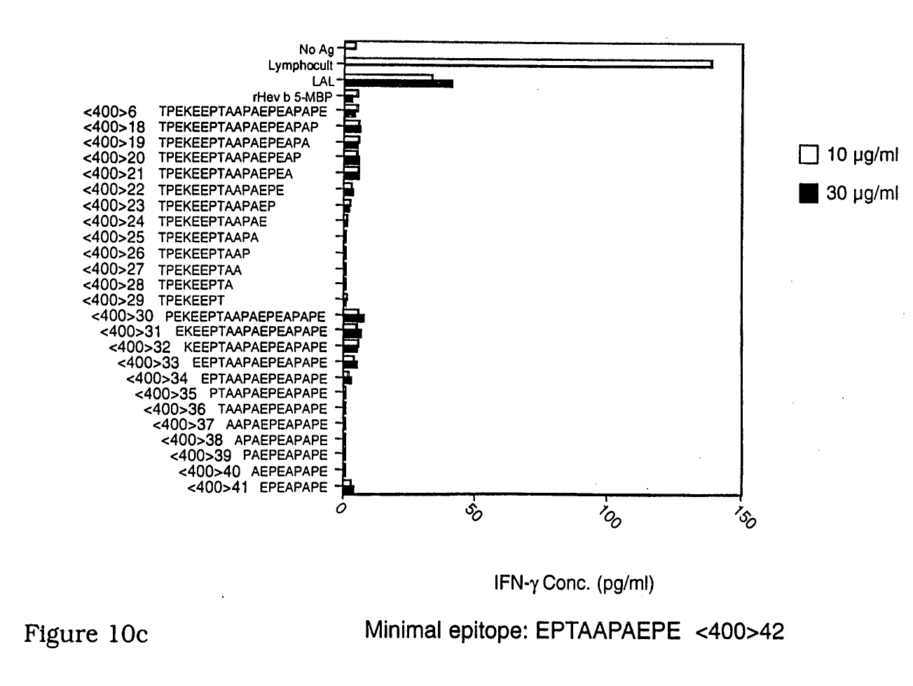

The mice generated a strong polyclonal immune response to rHev b 5/MBP, MBP and NA as demonstrated by ELISA (FIG. 6). The response to rHev b 5/MBP and NAL was stronger than that to MBP alone, indicating a specific immune response to rHev b 5. Five hybridomas with the desired antibody specificity (positive for 4rHev b 5/MBP but negative to MBP were generated). Three of these (1C10, 6F6 and 3G3) also recognised NAL strongly, the other 2 recognising NAL much less so (FIG. 7). Western blotting of rHev b 5/MBP digest showed that MoAb 1C10 detected rHev b 5/MBP and rHev b 5 but not MBP alone (FIG. 8). When blots of NAL were probed with MoAB 1C10, a protein band at 24 kDa was strongly detected, while a band at 80 kDa was also detected, though much less so (FIG. 9)

### EXAMPLE 3

### B Cell Epitope Mapping of Hev b 5

It is noteworthy that, unlike some other major allergens, the amino acid sequence of Hev b 5 lacks cysteine residues, which are involved in disulphide bonds and thus stabilisation of the tertiary structure of a protein. This indicates that some of the IgE epitopes of Hev b 5 are linear or continuous, which makes the synthetic peptide approach of epitope mapping useful, as has been previously found in Lol p 5 (Suphioglu et al. 1998).

Synthetic overlapping peptides based on the deduced amino acid sequence of the mature Hev b 5 protein (fourteen 20-mers with 11 amino acid overlap) are be used to identify linear B cell epitopes following established protocols (Suphioglu et al. 1998). Briefly, synthetic peptides are immobilised either on nitrocellulose membranes or microtitre plates, non-binding sites blocked with 3% BSA/PBS and IgE-binding of patients' sera detected with mouse anti-human IgE followed by horse radish peroxidase conjugated anti-mouse antibodies. Total non-ammoniated latex (NAL) and glove extract (GE) proteins and recombinant Hev b 5 (recHev b 5) are immobilised also, as positive controls. IgE reactive Hev b 5 peptides, along with NAL, GE proteins and recHev b 5, as positive controls, are evaluated for their capacity to inhibit IgE binding to the natural and recHev b 5 allergens. This indicates whether or not the identified IgE-reactive peptides correspond to IgE-reactive epitopes revealed within the context of the natural and recHev b 5 allergens.

Conformational B cell epitopes of Hev b 5 (if any) are identified with random mutagenesis. In this strategy, the plasmid vector containing the cDNA encoding IgE-reactive recombinant Hev b 5 is transformed into XL1-Red host cells (Epicurian Coli, Stratagene, CA, USA) to generate random mutations within the cDNA encoding Hev b 5. The XL1-Red strain is deficient in three of the primary DNA repair pathways in E. coli (ie. mutS, mutD and mutT) making its mutation rate approximately 5,000-fold higher than that of its wild-type parent. After transformation and propagation in XL1-Red strain, plasmid DNA is retrieved and transformed into XL1-Blue strain for analysis. This is necessary as every time the plasmid replicates in the XL1-Red strain, further mutations occur. Once transformed into the XL1-Blue, different dilutions of the transformants are plated to obtain single colonies for immunoscreening with IgE scra (which has been preabsorbed with E. coli extract) known to possess recHev b 5-specific IgE. Immunoscreening of bacterial colonies identifies mutants defective in IgE-binding, as compared to non-mutated recHev b 5 control, and responsible mutations will be resolved by DNA sequencing. Such B cell epitope sequences are compared with the linear B cell epitopes identified above to determine if conformational epitopes have been resolved.

Those skilled in the art will appreciate that the invention described herein is susceptible to variations and modifications other than those specifically described. It is to be understood that the invention includes all such variations and modifications. The invention also includes all of the steps; features, compositions and compounds referred to or indicated in this specification, individually or collectively, and any and all combinations of any two or more of said steps or features.

TABLE 3
Clinical characteristics of latex allergic patients

| | | | | | | Latex EAST score out |
|---|---|---|---|---|---|---|
| | | | | Clinical food | Clinical kiwi fruit | of 4 (assay result in |
| Patient | Age | Sex | Clinical symptoms on contact with latex | allergy | allergy | AEU/ml) |
| 1 | 37 | F | Urticaria, asthma, angioedema, anaphylaxis | Yes | No | 4 (16.4) |
| 2 | 55 | F | Urticaria, angioedema | Yes | Yes | 3 (7.1) |
| 3 | 41 | F | Urticaria, rhinitis, asthma, angioedema, anaphylaxis | Yes | Yes | 3 (9.5) |
| 4 | 65 | F | Urticaria, rhinitis, asthma, angioedema, anaphylaxis | Yes | Yes | 4 (>17.5) |
| 5 | 30 | F | Urticaria, angioedema | Yes | Unknown | 4 (>17.5) |
| 6 | 41 | F | Urticaria, rhinitis, asthma, angioedema, anaphylaxis | Yes | Unknown | 3 (9.5) |

TABLE 4
Oligoclonal proliferative responses to
Hev b 5 peptides of latex allergic donors

| Antigen | | P1 | P2 | P3 | P4 | P5 | P6 |
|---|---|---|---|---|---|---|---|
| T + APC | alone | 3.6 | 2.1 | 5.2 | 0.9 | 1.0 | 1.0 |
| LAL | 100 | 9.5 | 12.6 | 55.9 | 2.2 | 4.1 | 29.9 |
| | 30 | 7.8 | 9.8 | 65.9 | 2.9 | 4.6 | 27.9 |
| | 10 | 8.8 | 9.4 | 63.4 | 3.1 | 3.3 | 14.6 |
| | 3 | 7.1 | 7.7 | 37.1 | 2.0 | 1.7 | 9.9 |
| Hey b 5-MBP | | 9.0 | 5.7 | NT | NT | NT | NT |
| MBP | | 2.5 | 1.5 | NT | NT | NT | NT |
| (1-20) | 30 | 2.5 | 2.1 | 8.2 | 2.0 | 2.6 | 2.2 |
| | 10 | 3.0 | 1.6 | 7.6 | 1.6 | 0.9 | 1.0 |
| (10-29) | 30 | 3.3 | 1.7 | 5.2 | 1.2 | 0.8 | 1.7 |
| | 10 | 3.2 | 1.4 | 7.7 | 1.6 | 1.5 | 1.1 |
| (19-38) | 30 | 2.4 | 2.4 | 3.0 | 1.6 | 4.0 | 2.0 |
| | 10 | 2.4 | 1.1 | 6.4 | 1.2 | 0.6 | 0.9 |
| (28-47) | 30 | 5.0 | 41.7 | 6.9 | 1.4 | 1.6 | 1.1 |
| | 10 | 7.2 | 25.8 | 6.4 | 1.2 | 0.8 | 0.9 |
| (37-56) | 30 | 8.5 | 38.8 | 2.7 | 3.2 | 1.1 | 0.9 |
| | 10 | 7.1 | 18.3 | 8.8 | 3.0 | 1.4 | 1.4 |
| (46-65) | 30 | 19.6 | 22.6 | 19.2 | 5.3 | 5.6 | 1.2 |
| | 10 | 21.3 | 13.4 | 25.8 | 4.4 | 4.9 | 1.5 |
| (55-74) | 30 | 7.7 | 5.6 | 8.2 | 2.8 | 0.7 | 0.9 |
| | 10 | 5.0 | 2.8 | 9.1 | 2.0 | 1.1 | 0.7 |
| (64-83) | 30 | 5.6 | 2.1 | 4.3 | 2.6 | 2.4 | 1.0 |
| | 10 | 3.5 | 1.8 | 7.0 | 1.9 | 0.5 | 0.8 |
| (73-92) | 30 | 2.9 | 14.6 | 3.1 | 1.2 | 0.5 | 1.3 |
| | 10 | 2.5 | 9.8 | 5.3 | 1.2 | 0.8 | 1.0 |
| (82-101) | 30 | 5.2 | 2.1 | 6.5 | 2.6 | 1.6 | 1.2 |
| | 10 | 4.1 | 3.2 | 7.3 | 2.4 | 1.3 | 0.8 |
| (91-110) | 30 | 2.6 | 3.2 | 8.1 | 2.2 | 3.2 | 1.2 |
| | 10 | 2.4 | 2.4 | 7.4 | 1.6 | 1.1 | 0.7 |
| (100-119) | 30 | 2.0 | 2.2 | 8.0 | 1.1 | 0.8 | 1.0 |
| | 10 | 2.4 | 1.3 | 6.8 | 1.2 | 1.0 | 0.7 |
| (109-128) | 30 | 5.6 | 42.7 | 17.3 | 3.0 | 0.9 | 0.6 |
| | 10 | 8.2 | 36.9 | 13.7 | 2.3 | 1.3 | 0.9 |
| (118-137) | 30 | 2.5 | 20.8 | 8.5 | 1.2 | 1.3 | 0.8 |
| | 10 | 3.9 | 19.0 | 6.8 | 0.9 | 0.5 | 0.9 |
| (127-146) | 30 | 4.4 | 6.6 | 8.3 | 1.6 | 2.5 | 0.8 |
| | 10 | 3.3 | 3.8 | 7.1 | 2.2 | 2.7 | 1.0 |
| (132-151) | 30 | 6.5 | 3.3 | 7.0 | 1.7 | 1.0 | 0.8 |
| | 10 | 4.1 | 2.7 | 6.7 | 1.1 | 1.4 | 1.2 |

Latex specific 3-week T cell lines from latex allergic patients (P1-P6) were stimulated with the Hey b 5 peptides at 10 and 30 μg/ml, LAL at 3, 10, 30 and 100 μg/ml and rHev b 5/MBP at 10 μg/ml in 4 day cultures. The responses for each peptide and protein antigen are given as counts per minute × 10⁻³ and compared with the control response where T cells and APC were incubated alone in the absence of antigen.
Stimulation indices of ≧2.5 are shown in bold type.
NT = not tested.

TABLE 5
IL-5 and IFNγ levels produced by latex specific T cell lines when
stimulated with Hev b 5 peptides

| | Hev b5 Peptides | IL-5 | | IFNγ | | IL-5/IFNγ | |
|---|---|---|---|---|---|---|---|
| | tested | (pg/ml) | | pg/ml) | | ratio | |
| Patient | (30, 10 μg/ml) | 30 | 10 | 30 | 10 | 30 | 10 |
| P1 | 28-47 (−) | 68 | 0 | 0 | 0 | — | — |
| | 37-56 (−) | 0 | 0 | 0 | 0 | — | — |
| | 46-65 (+) | 279 | 344 | 2 | 2 | 139.5 | 172 |
| | 55-74 (−) | 0 | 0 | 0 | 0 | — | — |
| | 64-83 (−) | 0 | 14 | 0 | 0 | — | — |
| | 73-92 (−) | 0 | 0 | 0 | 0 | — | — |
| | 100-109 (−) | 0 | 0 | 0 | 0 | — | — |
| | 109-128 (−) | 69 | 0 | 0 | 0 | — | — |
| P2 | 28-47 (+) | 678 | 706 | 112 | 122 | 6.1 | 5.8 |
| | 37-56 (+) | 767 | 744 | 77 | 74 | 10 | 10.1 |
| | 46-65 (+) | 408 | 58 | 54 | 18 | 7.6 | 3.2 |
| | 55-74 (−) | 0 | 0 | 4 | 2 | — | — |
| | 64-83 (−) | 0 | 0 | 4 | 2 | — | — |
| | 73-92 (+) | 287 | 141 | 45 | 18 | 6.4 | 7.8 |
| | 100-119 (−) | 17 | 0 | 4 | 2 | 4.2 | — |
| | 109-128 (+) | 595 | 293 | 97 | 73 | 6.1 | 4 |
| P3 | 46.65 (+) | 3186 | 3799 | 227 | 388 | 14 | 9.8 |
| | 64-83 (−) | 205 | 109 | 129 | 132 | 1.6 | 0.8 |
| | 73-92 (−) | 24 | 27 | 83 | 112 | 0.3 | 0.2 |
| | 109-128 (+) | 1559 | 1403 | 292 | 257 | 5.3 | 5.5 |
| P4 | 37-56 (+) | 296 | 173 | 6 | 0 | 49.3 | 173 |
| | 46-65 (+) | 519 | 561 | 12 | 25 | 43.2 | 22.4 |
| | 55-74 (+) | 199 | 70 | 0 | 0 | 199 | 70 |
| | 64-83 (+) | 56 | 0 | 4 | 0 | 14 | 0 |
| | 82-101 (+) | 280 | 285 | 0 | 0 | 280 | 285 |
| | 100-119 (−) | 0 | 0 | 0 | 0 | — | — |
| | 109-128 (+) | 212 | 166 | 0 | 0 | 212 | 166 |
| | 118-137 (−) | 0 | 0 | 0 | 0 | — | −1 |
| P5 | NotTested | | | | | | |

Supernatants were removed at 48 hours from T cell cultures and tested in an ELISA for the presence of IL-5 and IENγ. Proliferation inducing and non-inducing peptides are indicated by (+) and (−) respectively. The levels of IL-5 and IFNγ produced in pg/ml are shown at peptide concentrations of 30 and 10 μg/ml. Where both cytokines were detectable the IL- 5/IFNγ ratio is shown at peptide concentrations of 30 and 10 μg/ml.

### BIBLIOGRAPHY

Akasawa A et al; “A novel acidic allergen, Hev b 5, in latex. Purification, cloning and characterization”; J Biol Chem 1996; 271:25389-25393.
Amann et al; (1988); Gene 69:301-315.
Baldari et al (1987); Embo J. 6:229-234.
Brehler R et al; “Latex-fruit syndrome—frequency of cross-reacting IgE antibodies”; Allergy 1997; 52:404-410.
Breitender H; “The Allergens of Hevea brasiliensis”; Allergy Clin Immunol Int 1998; 10:101-109.
Brown R H et al; “Prevalence of latex allergy among anaesthesiologists: identification of sensitized but asymptomatic individuals”; Anaesthesiology 1998; 89:292-299.
Brugnami G et al; “Work-related late asthmatic response induced by latex allergy”; J Allergy Clin Immunol 1995; 96:457-464.
Chen Z et al; “Allergenic and antigenic determinants of latex allergen Hev b 1: peptide mapping of epitopes recognized by human, murine and rabbit antibodies”; Clin Exp Allergy 1996; 26:406-415.
Chen Z et al; “Identification of hevein (Hev b 6.02) in Hevea latex as a major cross-reacting allergen with avocado fruit in patients with latex allergy”; J Allergy Clin Immunol 1998; 102:476-481.
Douglas R et al; “Prevalence of IgE-mediated allergy to latex in hospital nursing staff”; Aust N Z J Med 1997; 27:165-169.
Goding, J. Monoclonal Antibodies: principles and practice 3rd ed. London: Academic Press. 492, (1996).
Grzybowski M et al; “The prevalence of anti-latex IgE antibodies among registered nurses”; J. Allergy Clin Immunol 1996; 98:535-544.
Jameel et al (1990); J. Virol. 64:3963-3966.
Kibby T and Akl M; “Prevalence of latex sensitisation in a hospital employee population”; Ann Allergy Asthma Immunol 1997; 78:41-44.
Knapp et al (1990); Bio Techniques 8:280-281.
Köhler, G. and Milstein, C. Nature 256(5517):495-7 (1975).
Kostyal D A et al; “Cloning and characterization of a latex allergen (Hev b 7): homology to patatin, a plant PLA2”; Clin Exp Immunol 1998; 112:355-362.
Kurjan and Herskowitz (1982); Cell 30:933-943.
Liss G M et al; “Latex allergy: epidemiological study of 1351 hospital workers”; Occ Environ Med 1997; 54:335-342.
O'Hehir R E et al; “Cloned human T lymphocytes as reactive with Dermatophagoides farinae (house dust mite): a comparison of T and B cell antigen recognition”. Immunology 1987; 62:635-640.
O'Hehir R E et al; “House dust mite allergy: from T cell epitopes to immunotherapy”; Eur J Clin Invest 1993; 23:763-772.
Ownby D R et al; “Anaphylaxis associated with latex allergy during barium enema examinations”; AJR. 1991; 156:903-908.
Posch A et al; “Latex allergens”; Clin Exp Allergy 1998; 28:134-140.
Raulf-Heimsoth M et al; “Analysis of T-cell reactive regions and HLA-DR4 binding motifs on the latex allergen Hev b 1 (rubber elongation factor)”; Clin Exp Allergy 1998; 28:339-348.
Sambruck et al; “Molecular cloning: a laboratory manual, Second Edition”; Cold Spring Harbour Laboratory Press; Cold Spring Harbour, N.Y.; 1989.
Scheiner O et al; “Cloning and molecular characterization of Hev b 3, a spina-bifida-associated allergen from Hevea brasiliensis latex”; Int Arch Allergy Immunol 1999; 118:311-312.
Schultz et al (1987); Gene 54:113-123.
Slater J E et al; “Murine B-cell and T-cell epitopes of the allergen Hev b 5 from natural rubber latex”; Mol Immunol 1999; 36:135-143.
Slater J E et al; “Identification, cloning, and sequence of a major allergen (Hev b 5) from natural rubber latex (Hevea brasiliensis)”; J Biol Chem 1996; 271:25349-24399.
Slater J E; “Latex allergy”: J Allergy Clin Immunol 1994; 94:139-149.
Slater, J. E. et al. Journal of Biological Chemistry 271(41):25389-93 (1996).
Sowka S et al; “cDNA cloning of the 43-kDa latex allergen Hev b 7 with sequence similarity to patatins and its expression in the yeast Pichia pastoris”; Eur J Bioch 1998; 255:213-219.
Suphioglu C et al; “Identification of canary grass (Phalaris aquatica) pollen allergens by immunoblotting: IgE and IgG antibody binding studies”; Allergy 1993; 48:273-281.
Suphioglu, C., Blaher. B., Rolland, J. M. McCluskey, J., Kenrick, J. Schäppi, G., Singh, M. B. and Knox, R. B. Molecular basis of IgE-recognition of Lol p 5, a major allergen of rye-grass pollen. Molecular Immunology 1998; 35:293-305.
Sussman G L et al; “The spectrum of IgE-mediated responses to latex”; JAMA 1991; 265:2844-2847.
Varney et al, British Medical Journal, 302:265-269 (1990).
Yeang H. Y. et al; “The 14.6 kd rubber elongation factor (Hev b 1) and 24 kd (Hev b 3) rubber particle proteins are recognized by IgE from patients with spina bifida and latex allergy”; J Allergy Clin Immunol 1996; 98:628-639.

What is claimed is:

1. A method for the treatment and/or prophylaxis of a condition in a subject, which condition is characterized by an aberrant, unwanted or otherwise inappropriate immune response to Hev b 5, said method comprising administering to said subject an effective amount of a peptide comprising a Hev b 5 T cell epitope, said peptide comprising at least 5 contiguous amino acids of an amino acid sequence selected from the group consisting of:
(i) amino acids 1-20;
(ii) amino acids 37-56;
(iii) amino acids 46-65;
(iv) amino acids 55-74;
(v) amino acids 82-101;
(vi) amino acids 109-128;
(vii) amino acids 127-146; and
(viii) amino acids 132-151
of Hev b 5 (SEQ ID NO: 1) or a homolog or mutant of said peptide, for a time and under conditions sufficient to remove or reduce the presence or function in said subject of T cells directed to said Hev b 5, wherein said peptide is capable of interacting with T cells and modifying T cell function when incubated with cells from human subjects having a condition characterized by an aberrant, unwanted or otherwise inappropriate immune response to Hev b 5 and said peptide is unable to undergo IgE binding.

2. The method according to claim 1, wherein said condition is latex hypersensitivity.

3. The method according to claim 1, wherein said peptide is from 5 to 20 residues in length.

4. The method according to claim 3, wherein said amino acid sequence is amino acids 1-20 inclusive of Hev b 5.

5. The method according to claim 3, wherein said amino acid sequence is amino acids 37-56 inclusive of Hev b 5.

6. The method according to claim 3, wherein said amino acid sequence is amino acids 46-65 inclusive of Hev b 5.

7. The method according to claim 3, wherein said amino acid sequence is amino acids 55-74 inclusive of Hev b 5.

8. The method according to claim 3, wherein said amino acid sequence is amino acids 82-101 inclusive of Hev b 5.

9. The method according to claim 3, wherein said amino acid sequence is amino acids 109-128 inclusive of Hev b 5.

10. The method according to claim 3, wherein said amino acid sequence is amino acids 127-146 inclusive of Hev b 5.

11. The method according to claim 3, wherein said amino acid sequence is amino acids 132-151 inclusive of Hev b 5.

12. The method according to claim 3, wherein said peptide comprises at least 5 consecutive amino acids from one or more of the following amino acid sequences:

| | | |
|---|---|---|
| (i) | MASVEVESAATALPKNETPE | (SEQ ID NO:2) |
| (ii) | ASEQETADATPEKEEPTAAP | (SEQ ID NO:6) |
| (iii) | TPEKEEPTAAPAEPEAPAPE | (SEQ ID NO:7) |
| (iv) | APAEPEAPAPETEKAEEVEK | (SEQ ID NO:8) |
| (v) | APEADQTTPEEKPAEPEPVA | (SEQ ID NO:11) |
| (vi) | TKETETEAPAAPAEGEKPAE | (SEQ ID NO:14) |
| (vii) | AEEEKPITEAAETATTEVPV | (SEQ ID NO:16) |
| (viii) | PITEAAETATTEVPVEKTEE. | (SEQ ID NO:17) |

13. The method according to claim 12, wherein said amino acid sequence is from one or more of SEQ ID NO:7 or SEQ ID NO:14.

14. The method according to claim 13, wherein said amino acid sequence is from SEQ ID NO:7.

15. The method according to claim 14, wherein the peptide comprises SEQ ID NO:42.
